(19) United States
(12) Patent Application Publication
(10) Pub. No.: US 2024/0180452 A1
(43) Pub. Date: Jun. 6, 2024

(54) MULTI-SPECTRAL IMAGING SYSTEMS FOR ASSESSING HEALTH

(71) Applicant: NXgenPort Inc, St. Paul, MN (US)

(72) Inventors: Mohamed M. Ali, St. Paul, MN (US); Rosanne Welcher, Ventura, CA (US); Cathy Skinner, St. Paul, MN (US); Nancy Lelicoff, Fair Oaks, CA (US)

(21) Appl. No.: 18/286,277

(22) PCT Filed: Apr. 13, 2022
(86) PCT No.: PCT/US2022/024635

§ 371 Date: Oct. 10, 2023

Related U.S. Application Data
(60) Provisional application No. 63/268,476, filed on Feb. 24, 2022.
(60) Provisional application No. 63/268,474, filed on Feb. 24, 2022.
(60) Provisional application No. 63/268,480, filed on Feb. 24, 2022.
(60) Provisional application No. 63/268,477, filed on Feb. 24, 2022.
(60) Provisional application No. 63/174,319, filed on Apr. 13, 2021.

(51) Int. Cl. A61B 5/145; A61B 5/00; A61B 5/01; A61B 5/02; A61B 5/0205; A61B 5/0215; A61B 5/0245; A61B 5/026; A61B 5/029; A61B 5/1455; A61B 5/1459; G16H 10/20; G16H 15/00; G16H 20/17; G16H 50/20

(52) U.S. Cl. CPC A61B 5/14546; A61B 5/0006; A61B 5/0008; A61B 5/0022; A61B 5/02055; A61B 5/6852; A61B 5/6876; A61B 5/7246; A61B 5/7275; A61B 5/746; G16H 10/20; G16H 15/00; G16H 20/17; G16H 50/20; A61B 5/01; A61B 5/02028; A61B 5/0215; A61B 5/0245; A61B 5/0261; A61B 5/029; A61B 5/14552; A61B 5/1459; A61B 5/443; A61B 5/4845; A61B 2503/04; A61B 2503/06; A61B 2503/42; A61B 2560/045; A61B 2562/0233; A61B 2562/0271; A61B 2562/046

(57) ABSTRACT

This disclosure provides implantable devices with remote physiological monitoring capabilities. In particular, this disclosure relates to implantable devices with multi-spectral sensors that sense analyte from within a subject to provide clinically actionable data related to the subject's heath. For example, in preferred embodiments, the devices relate to ports with one or more multi-spectral sensors capable of remotely measuring symptoms of physiological distress via the multi-spectral sensors. In some embodiments, the ports are useful to deliver chemotherapy treatments and monitor patient health over the course of chemotherapy treatments. The multi-spectral sensors sense physical light properties modulated (e.g., reflected, absorbed, dispersed, scattered, etc.) by one or more analytes present in the body to allow for earlier detection of signs indicative of a health status.

### RELATED APPLICATIONS

The present application claims the benefit of and priority to U.S. provisional application Ser. No. 63/174,319, filed Apr. 13, 2021, U.S. provisional application Ser. No. 63/268,476, filed Feb. 24, 2022, U.S. provisional application Ser. No. 63/268,474, filed Feb. 24, 2022, U.S. provisional application Ser. No. 63/268,480, filed Feb. 24, 2022, and U.S. provisional application Ser. No. 63/268,477, filed Feb. 24, 2022. The content of each of which is incorporated herein by reference in its entirety.

### TECHNICAL FIELD

This disclosure relates to implantable medical devices having one or more multi-spectral sensors for assessing a health status in a patient or subject such as an adult or pediatric patient, and animal or a clinical trial subject.

### BACKGROUND

Patients with chronic illnesses (e.g., cancer) make frequent visits to healthcare facilities. The purpose of these visits may be for routine treatments and/or health assessments that provide a snapshot of the patient's health. One common assessment involves a blood analysis, which is useful for evaluating treatment efficacy and/or identifying potential side effects of a drug.

To facilitate frequent treatments, some patients are provided with a port-catheter, i.e., a port. Ports are small medical devices implanted under the patient's skin to provide convenient access to the circulatory system for drug delivery. Ports generally do not require any maintenance. However, ports do occasionally fail, break, or leak fluid. As such, ports must be routinely inspected at healthcare facilities to ensure proper function.

However, for many patients, visiting a healthcare facility is unduly difficult. Some patients, for example, must commute long distances to reach the nearest healthcare facility. And depending on the severity of a patient's condition, commuting may require personal assistance, which is not always available. Thus, many patients fail to show up for scheduled appointments and/or are rarely monitored in between treatments. As such, physicians are unable to determine whether a treatment is working or if the treatment is causing harmful side effects, such as organ damage. As such, complications associated with a failed or leaking port may go undetected.

### SUMMARY

This disclosure provides implantable devices (e.g., ports) with remote monitoring capabilities for assessing health in adult and pediatric patients, generating research or health status data in animals, and/or obtaining clinical data and health status of a human clinical trial subject. The implantable devices include one or more multi-spectral sensors that enable optical detection of one or more analytes inside the subject's body. For example, in preferred embodiments, the devices relate to ports with one or more multi-spectral sensors capable of remotely measuring symptoms of physiological distress (e.g., fever, heart rate aberrations, blood cell count fluctuations) via the multi-spectral sensors. In some embodiments, the ports are useful to deliver chemotherapy treatments and monitor patient health over the course of chemotherapy treatments. The multi-spectral sensors sense physical light properties modulated (e.g., reflected, absorbed, dispersed, scattered, etc.) by one or more analytes present in the body to allow for earlier detection of signs indicative of a health status. This is useful for, among other things, early detection of complications associated with implantable ports, e.g., infections or thrombosis, and to evaluate patients prior to a scheduled chemotherapy treatment.

Moreover, devices of the invention may be equipped with wireless communication components that provide for remote transmission of clinically actionable data to one or more locations, such as, one or more treatment facilities. Accordingly, a health status of the subject can be periodically reported to the subject's treating physician without the subject ever leaving the safety and comfort of their own home. Moreover, whereas conventional health assessments only take snapshots of a subject's health while present at a treatment facility, devices of the invention continue to monitor subject health remotely and frequently and thus can more precisely identify when a subject needs a treatment or is unfit to receive a particular treatment. As such, devices of the invention may enable earlier detection of potentially severe medical problems including, for example, infection or heart aberrations. These devices can send an alert to the subject and/or the subject's treating physician when a symptom of a potentially severe problem is detected.

Devices of the invention provide data using in vivo sensing of one or more analytes present in blood to support remote patient monitoring. By monitoring the one or more analytes remotely, unnecessary laboratory visits may be prevented. For example, devices of the invention may identify that a patient has a poor blood count in advance of a scheduled chemotherapy treatment, and as such, is unfit to receive a scheduled chemo treatment. Accordingly, devices of the invention may reduce workload on clinical laboratories and improve treatment efficiency. Moreover, many chronically ill subjects may have compromised immune systems, either because of their illness or immuno-suppressive effects of treatment. Therefore, it is advantageous to avoid unnecessary facility visits and contact with potential sources of infection such as might be found in waiting rooms or by commuting to a treatment facility. By making measurements in vivo with the implantable devices of the invention, visits to the treatment facilities are only made when such a visit is warranted and avoided in instances where a condition might prevent the patient from receiving treatment.

It is an insight of the invention that one or more analytes (e.g., cells, nucleic acids, proteins, etc.) exhibit characteristic light absorption and scattering properties that are unique to clinically relevant information (e.g., size, cellular content, molecular structure) of the analytes. Devices and methods of the invention take advantage of these unique properties characterize one or more analytes in a subject and, based on the characterizations, assess a health status of a subject. The health assessments are useful to identify early signs of infection, changes in heart function, blood cell counts, oxygen levels, body temperature, proteins (i.e., hemoglobin), cancer recurrence/monitoring, and/or device leakage or failure.

Accordingly, in one aspect, this disclosure relates to an implantable device comprising a multi-spectral imaging sensor system. The multi-spectral imaging sensor system senses characteristic light absorption and scattering properties of one or more analytes to provide for in vivo detection of tissue (e.g., blood) parameters including physiology, morphology, and/or composition. Parameters measured from a subject can be correlated by computer algorithms, e.g., machine learning algorithms, with parameters associated with a known patient health status to provide reliable information useful for monitoring the subject's health.

Multi-spectral imaging sensors of the invention are dimensioned for implantation into blood vessels, via a port-catheter, to assess one or more analytes present in a subject's blood stream. Preferably, the multi-spectral imaging sensor is provided as part of a system that includes a spectrum-resolving component, for example, at least one of a filter, a grating, or a prism. The spectrum-resolving component is operable to distinguish a plurality of distinct wavelengths of light, for example, wavelengths of light between about 400 and 2000 nanometers. The absorption, reflection, and scattering of light at multiple different wavelengths, in combination with an algorithm, e.g., a machine learning algorithm trained on data to recognize and characterize properties of analytes in blood based on flow dynamics, may provide a helpful clinical assessment of a subject's health status.

Moreover, some analyte of interest may exhibit autofluorescence signatures attributable to, for example, protein and/or nucleic acid content and/or the distribution of such composites within the analyte. Accordingly, devices of the invention may leverage detection of autofluorescence to generate a more complete health assessment of a subject. As such, in some instances, devices of the invention are further equipped with at least one additional component operable to sense autofluorescence.

In preferred embodiments, the multi-spectral imaging sensor system is provided as part of a catheter, e.g., a port. A port relates to an implantable device, typically intended for use more than 30 days, which is placed under the skin of a subject. The port provides a point of entry to a subject's central venous system for periodic delivery of treatments (e.g., chemotherapy agents). The presence of the port eliminates the need for repeated needle insertions into a subject's small blood vessels of the arms or hands, which often leads to scarring, blood vessel narrowing, or blood vessel collapse. As such, according to some preferred embodiments, devices of the invention combine advantages of port-catheter with optical sensors to measure, monitor, and report on physiological functions of a subject's body, in vivo, over the course of disease and treatment.

Accordingly, medical devices of the invention may relate to a catheter system. The catheter system may be designed to remain in place for at least one week to monitor patient health and/or assess a treatment efficacy. The catheter system may include a cannula with a proximal portion and a distal portion. Preferably, the multi-spectral imaging sensor system is attached to the distal portion of the cannula. For example, the multi-spectral imaging sensor system may be disposed at the distal tip of the cannula, or, more preferably, at a distal portion of the cannular adjacent to the distal tip. Advantageously, by positioning the multi-spectral sensor system at a distal portion adjacent to the tip, as opposed to at the tip, dislodging or otherwise harming fragile components of the sensor, which are prone to damage, during insertion or retrieval of the catheter system is avoided. The catheter device may further include a port having a self-sealing septum for receiving fluid (e.g., a chemotherapy agent) via a needle.

In some embodiments, devices of the invention include an assembly of components for detection, analysis, and transmission of clinically relevant data. For example, devices of the invention may include a two-dimensional photosensor array and a fiber optic image conduit. The fiber optic image conduit may be operable to relay data from the multi-spectral imaging sensor system to the two-dimensional photosensor array. Preferably, the two-dimensional photometric sensor array is operable to acquire data at a high sampling rate of more than 10 frames per second. Devices may further include a broad-spectral light source and a communication module operable to provide data to a computing device that is external to the subject.

In another aspect, this disclosure relates to a method for monitoring patient health. The method may be useful to monitor health status of chronically ill patients remotely. For example, the method may be useful to identify when a chronically ill patient is in need of a treatment without burdening the patient with making a long commute to a treatment facility.

Advantageously, these methods alleviate problems associated with frequent inpatient examinations, which are often missed, and provide for early detection of potential health issues.

Methods of the invention include implanting a device into a subject. The device includes a multi-spectral imaging sensor system configured to sense an analyte (e.g., a blood cell, a circulating tumor cell, a protein, a microbe or a nucleic acid etc.) in the subject. Sensing of the analyte by the sensor is used to assess a health status, preferably remotely, of the subject.

The multi-spectral imaging sensor is capable of sensing one or more of light dispersion, scattering, light diffraction, or light interference of one or more analytes. By sensing one or more of these light properties, the multi-spectral imaging sensor is useful to assess one or more of size, granularity, nuclear size, shape, or cytoplasmic density, of the one or more analytes. Preferably, the multi-spectral imaging sensor comprises spectrum-resolving components, for example, at least one of a filter, a grating, or a prism, for sensing a plurality of distinct wavelengths of light between, for example, 400 and 2000 nanometers. By sensing a plurality of distinct wavelengths of light and, for example, analyzing the wavelengths of light with an algorithm trained to recognize and characterize flow patterns of one or more analytes in blood, methods of the invention are useful to collect information relating to multiple properties of one or more analytes to thereby generate a more comprehensive report of a subject's health status.

The device, once implanted, may immediately begin collecting data. The data collected by the device may include information related to, for example, red blood cells, white blood cells, platelets, circulating tumor cells, microbes, nucleic acids, organic compounds, chemicals, chemical composition, drugs and/or hemodynamics, and cardiac function.

These data, as discussed in detail below, are useful for detection of early signs of infection, such as, thrombosis, or conditions related to treatment and/or disease. These data may be useful to indicate when a patient should follow up with additional lab tests, and in some instances, provide helpful clinical information for determining which tests should be conducted.

Preferably, the implanted device includes at least one additional sensor for sensing autofluorescence as some analyte of interest may exhibit autofluorescence signatures attributable to, for example, protein and/or nucleic acid content, which is useful to reveal clinically important information of a subject.

In preferred embodiments, the device comprises a catheter, e.g., a port-catheter. The catheter may include a port connected to a reservoir for receiving (or withdrawing) fluid by a needle. The fluid may be a drug, such as, a chemotherapy agent or an antibiotic.

The device, when implanted, may extend into at least one of a superior vena cava or a right atrium of the subject. Alternatively, without limitation, the device may extend into one of a peripheral vein or artery, central vein or artery, internal jugular vein or artery, subclavian vein or artery, axillary vein or artery, or a femoral vein or artery.

Accordingly, methods of the invention are useful for collecting data from one or more analytes in a natural, unperturbed state. The collection of such data is otherwise inaccessible. Accordingly, in vivo data collection by methods of the invention provide helpful clinical data, of high quality, and in some instances without concerns of contamination or artifacts, which are often introduced by conventional laboratory procedures.

According to another aspect, this disclosure provides a method of monitoring health of a subject that includes receiving, to a device, data based on light sensed by a multi-spectral imaging sensor system implanted in the subject. The data may be provided from a remote location by a wireless data network. The data may include, for example information related to one or more of red blood cells, white blood cells, platelets, circulating tumor cells, microbes, nucleic acids, chemicals, drugs and/or hemodynamics, and cardiac function, including blood flow rate and velocity and cardiac output, oxygen level, heart rate, body temperature, blood pressure, and ECG measurements of the patient.

Methods of the invention collect data that include multi-spectral light dispersion, scattering, absorption data of cells circulating in a blood stream of the subject. Methods further use algorithms, such as, machine learning algorithm trained to recognize and characterize analytes (e.g., blood cells) based on properties related to hemodynamics, to provide clinically relevant information about the subject.

Methods of the invention may further include analyzing data to generate an assessment of the subject's health. For example, analyzing may involve correlating data from the subject with one or more pre-determined parameters associated with a physiological condition. The physiological condition may be a chemotherapy related condition. The parameters may be selected based on input of a health care professional. For example, the parameters may be derived from data published in journal articles and/or based on measurements taken from a plurality (e.g., tens to hundreds to thousands) of different patients. Preferably, analyzing is performed using a machine learning system, as discussed in detail below. Accordingly, analyzing the data by methods described herein is useful to provide a clinically helpful information.

In some instances, the heath assessment may be automatically provided to medical personnel, e.g., a physician. For example, in some instances, if the subject is experiencing an adverse reaction, a data network may be used to provide an alert message to a healthcare provider immediately, via the secure communication link. And, based on the information provided by the alert message, the healthcare professional can manage the subject's health.

Certain aspects of the invention are useful to address a long-standing problem in the healthcare industry, which is that throughout patient treatment, the clinical status of a patient (e.g., a cancer patient) is largely unknown. Patient evaluations are often performed at clinical facilities prior to certain treatments, such as, chemotherapy treatments. These evaluations may be the first evaluation that the patient has received in an extended period of time (e.g., since the patient's last visit). Often, the evaluations reveal that it is unsafe to proceed with therapy due to uncontrolled symptoms, such as, low blood counts, or fever. This results in delays that can impact treatment efficacy and patient outcomes, while concurrently reducing utilization of cancer treatment unit facility time and nursing resources. Such delays, however, are avoidable by implementing devices provided by this disclosure. Additional, devices of the invention are useful to address problems associated with the rising volume of emergency room (ER) visits for cancer treatment-related toxicities when new or worsening symptoms emerge in between clinic visits. Such problems are well documented. For example, data show among such ER visits over a 10-year span, the most common and costliest complications diagnosed on presentation to ERs were neutropenia (9%, $5.5 billion), sepsis (8%, $11.2 billion), and anemia (8%, $6.8 billion). Of those ER visits, 91% resulted in inpatient admission to the hospital. Febrile neutropenia specifically can impact over 80% of patients with hematologic malignancies and a significant proportion of patients with solid tumors, and hospitalizations for these complications result in a prolonged hospital length of stay as well as higher healthcare costs compared with admissions for other reasons. Current ambulatory cancer treatment care models leave cancer patients unmonitored and sub-optimally supported for long periods while at risk of clinical deterioration. In one study, for example, looking at interval symptom monitoring, cancer patients receiving chemotherapy who were monitored with weekly symptom assessments facilitated by a web-based application were less frequently evaluated in an ER (34% v 41%) and remained on chemotherapy longer (mean, 8.2 v 6.3 months) than a similar group of patients receiving usual care without monitoring.

Devices of the invention address those issues by providing port-catheters enhanced with remote patient monitoring capabilities. For example, devices of the invention are useful to collect routine measurements of analytes in a patient between treatments to, among other things, facilitate early identification of adverse health symptoms and, in turn, enable early intervention. Accordingly, devices of the invention may improve patient quality of life and cancer treatment outcomes, and/or reduce ER visits and hospital admissions. As such, devices of the invention may reduce unnecessary patient travel and time away from home/work for testing and provider visits at medical facilities. Furthermore, in some embodiments, devices of the invention may enable dynamic cancer treatment scheduling to optimize operational efficiencies in cancer chemotherapy units, radiation suites, and operating rooms.

### BRIEF DESCRIPTION OF THE DRAWINGS

FIG. 1 shows an implantable device including a multi-spectral imaging sensor.

FIG. 2 shows a device with a multi-spectral imaging sensor in an axial configuration.

FIG. 3 shows a device with multi-spectral imaging sensors arranged in a circumferential configuration.

FIG. 4 shows a device with a multi-spectral imaging sensor equipped with a cone mirror.

FIG. 5 shows a device with multi-spectral sensors arranged in multiple configurations.

FIG. 6 shows an exemplary output graph produced by devices of the invention.

FIG. 7 shows a device implanted within a subject.

FIG. 8 shows an exemplary method for remote monitoring of patient health.

FIG. 9 shows an example of chemo-port in a pediatric subject.

FIG. 10 shows an example of tunneled catheter implanted in a neonate.

FIG. 11 shows an example of a peripherally inserted central catheter (PICC) line implanted in a pediatric subject.

FIG. 12a shows an exemplary method for shortening the length of catheters equipped with the disclosed sensor technology.

FIG. 12b. shows an alternate method for shortening the length of catheters equipped with the disclosed sensor technology.

FIG. 13 shows an exemplary method for remote monitoring of pediatric subjects.

FIG. 14 shows an example of chemo-port in a patient.

FIG. 15 shows examples of implantable intravenous access devices that may be used in patients undergoing various treatments for diseases.

FIG. 16 shows an exemplary method for remote monitoring of patients.

FIG. 17 depicts an example implantable device for use in animals.

FIGS. 18A, 18B, 18C, and 18D depict examples of device animal harnesses.

FIG. 19 depicts examples a device animal collar.

FIG. 20 depicts an exemplary method for remote monitoring of animal health.

FIG. 21 shows an exemplary method for remote monitoring of clinical trial subjects.

### DETAILED DESCRIPTION

This disclosure provides implantable devices having remote physiological monitoring capabilities. Devices of the disclosure include multi-spectral sensors that, when placed in contact with a subject's circulatory system, are capable of sensing one or more analytes to provide clinically actionable data related to the subject's heath status. In particular, devices of the invention interrogate tissue (e.g., blood) with short pulses of blue, green, red, or infrared light, or a broad spectral white light. The light is preferably delivered by one or more thin optical fibers of the implantable device. The light is manipulated (e.g., dispersed, scattered, absorbed, etc.) by one or more analytes present in tissue. The light manipulations may be sensed by a second fiber or bundle of fibers adjacent to the first fiber, to thereby collect data related to the one or more analytes and provide clinically useful information. The information may relate to one or more of red blood cells, white blood cells, platelets, circulating tumor cells, microbes, chemicals, drugs, nucleic acids, and/or hemodynamics, and cardiac function, including blood flow rate and velocity and cardiac output, oxygen level, heart rate, body temperature, blood pressure, and ECG measurements of the patient.

This information is useful to identify, among other things, early signs that a subject may be suffering from an infection and/or that a treatment is producing dangerous side effects (e.g., organ failure). In other instances, this information may be useful for showing signs of disease, for example, a genetic disease, an autoimmune disease, a neurologic disease, a metabolic disease, or a chemotherapy related condition.

For example, devices of the invention may collect data from one or more analytes circulating in blood which reveal clinically useful information relating to anemia or sickle cell disease. Anemia, for example, is a condition in which the body lacks sufficient healthy red blood cells to transport adequate oxygen to tissues. Signs of anemia may be detected by a complete blood cell count performed by devices of the invention. In instances of anemia, the complete blood cell count may reveal a low number of red blood cells (RBCs—as measured by the red cell count, the hematocrit, or the red cell hemoglobin content). In men, for example, the complete blood cell count may reveal evidence of anemia as defined by hemoglobin <14 g/dL (140 g/L), hematocrit <42% (<0.42), or RBC <4.5 million/mcL (<4.5×1012/L). In women, the complete blood cell count may reveal evidence of anemia as defined by hemoglobin <12 g/dL (120 g/L), hematocrit <37% (<0.37), or RBC <4 million/mcL (<4×10 12/L). In other instances, methods of the invention may detect signs of sickle cell disease. Sickle cell disease may be identified by the presence of the abnormal hemoglobin protein, referred to as hemoglobin S, which may distort red blood cells. In other instances, devices of the invention may reveal signs of a metabolic disease or a metabolic syndrome. A metabolic syndrome may be identified by high white blood cell content.

In certain embodiments, implantable devices of the invention provide for multi-spectral elastic light scattering spectroscopy of blood inside a subject's bloodstream. Elastic light scattering spectroscopy, according to devices of the invention, employs one or more fibers having a geometry that is sensitive to sub-cellular morphologies (e.g., sizes, shapes) and other features, such as, for example, nuclear grade and/or nuclear to cytoplasm ratio, mitochondrial size and density. In some embodiments, these features may correlate with features used by pathologists when performing a histological assessment. Accordingly, devices and methods of the invention are particularly useful for assessing conditions related to cancer.

For example, aneuploidy is one potential marker for assessing cancer. Aneuploidy is abnormal DNA content (any variation from the normal diploid number of chromosomes). At certain wavelengths, the most significant contribution to intensity data is scattering from cell organelles, and particularly the nucleus. Alterations in chromatin content, as occurs in aneuploidy, may give rise to localized changes in refractive index of subcellular components, which change the light spectra. As such, multi-spectral blood imaging, as provided by devices and methods of the invention, may be useful for monitoring a cancer patient by detecting, for example, and quantifying a percentage of cells having aneuploidy.

One aspect of the invention involves the insight that multi-spectral sensors may be integrated with implantable medical equipment (e.g., catheters) to report on one or more analytes present inside a subject during disease treatment. The multi-spectral sensors are useful to generate spectral signatures reflective of certain tissue parameters. These parameters can be correlated, preferably in real time, to parameters of, for example, heathy or diseased tissue, or to parameters of the subject previously measured (e.g., before treatment) to assess changes in a health status. For example, measurements of aneuploidy of a subject may be correlated with measurements of aneuploidy from the same subject before treatment. Correlating may involve aligning a data profile (e.g., detected light intensity or count over time) to a second data profile associated with a known health status (e.g., cancer). A decrease in the number of aneuploidy cells may reflect an improvement of the subject's health. An increase in the number of aneuploidy cells, however, may reflect a worsening (e.g., growing) of a cancer and thus trigger an alert to be sent to the treating physician.

Moreover, in preferred embodiments, the multi-spectral imaging sensors are integrated with catheters, e.g., a port-catheter. The port-catheter is dimensioned for complete implantation within a subject's body, e.g., placed under the skin of a subject. The port-catheter includes a reservoir with a self-sealing septum to provides a point of entry to a subject's central venous system for periodic delivery of treatments (e.g., chemotherapy agents). The presence of the port eliminates the need for repeated needle insertions into a subject's arms or hands, which often leads to unwanted medical complications. Accordingly, devices of the invention combine benefits associated with port-catheters and multi-spectral imaging sensors to take in vivo measurements over the course of a subject's treatment.

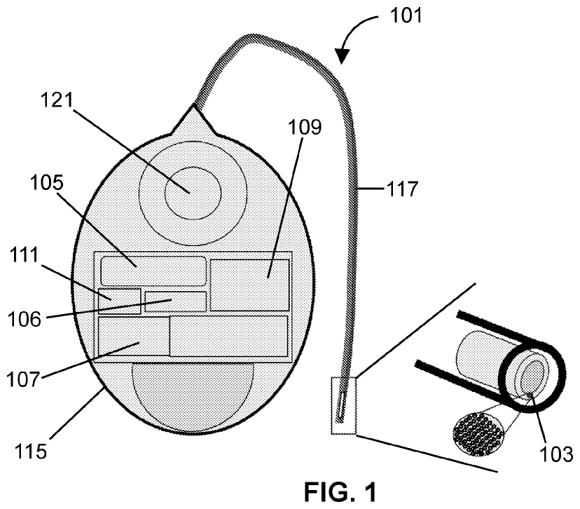

FIG. 1 shows an implantable device 101 with a multi-spectral imaging sensor 103.

The device 101 includes, among other things, a light source 105 (e.g., a light emitting diode), a spectrometer 106, a sensor/probe 103, a power supply 107, a computer 109 comprising memory to control various components and/or record measurements, and a communications module 111 to transmit data obtained from the multi-spectral imaging sensor 103 to a computing device external to the subject.

In some embodiments, the light source 105, spectrometer 106, and power supply 107 are substantially encased within one housing 115. The housing 115 may be made of a biocompatible metal (e.g., titanium), plastic, or polymer, e.g., polyether ether-ketone, or some combination thereof. The material may be selected for having biologically inert properties that allow the device to be implanted for at least one week and preferably longer, for example, at least one month, or at least two months, without eliciting an adverse reaction.

Preferably, the device 101 is dimensioned for surgical insertion under the skin of a subject. The housing 115 may be inserted, for example, in an upper chest region, or in an arm, of the subject. After insertion, the housing 115 may appear as a small bump under the skin. The device 101, once inserted, preferably requires no special maintenance.

The device 101 further includes a cannula 117 that may be surgically inserted into a blood vessel (e.g., into the jugular vein or artery, or subclavian vein or artery). Ideally, the cannula 117 terminates in the superior vena cava or the right atrium. As illustrated, the multi-spectral sensor system may be disposed at a distal portion of the cannula 117.

In preferred embodiments, devices of the invention are further equipped with at least one additional component operable to sense autofluorescence. Accordingly, in some embodiments, a distal portion of the cannula may include a sensor assembly including a plurality of photosensors. The plurality of photosensors may be arranged in an array format and configured to measure autofluorescence emitted by circulating analyte in combination with absorbance and or reflectance of light at one or more specific wavelengths.

In some embodiments, the device 101 comprises a fiber optic image conduit that relays, for example, a two-dimensional array of instantaneous light intensities to a two-dimensional photosensor array. The fiber optic image conduit may be disposed within the cannula 117. The two-dimensional photosensor array may comprise one or more of a complementary metal-oxide-semiconductor, a charge-coupled device, or photodiode arrays of adequate resolution so that each pixel of the fiber optic image conduit is represented by at least one pixel of the photometric sensor array. Preferably, the two-dimensional photometric sensor array is capable of acquiring light intensity information at a high sampling rate, for example, such as more than 10 frames per second. The high sampling rates allows for one or more analytes in fast moving blood to be readily detected and analyzed.

The device 101 may be constructed of discrete optical and optoelectronic components or integrated into an optical and optoelectronic construct, such as, for example, a micro-electro-mechanical system or a photonic integrated circuits-based sensor.

The light source 105 is configured to provide adequate intensity of light useful to illuminate an image field. Preferably the image field is within a blood vessel of a patient. The light source may be, for example, a laser, a super luminescent diode, a light emitting diode, or a wavelength tunable light source. The light may be delivered through the cannula 117 via a separate light guide, fiber optic cable, or may be coupled with an imaging fiber optic bundle via a beam splitter. In preferred embodiments, all optical components are constructed of high-quality optical grade material and include antireflection coatings, as needed, to increase the optical efficiency of the system and minimize stray light dispersion and reflection.

In at least one embodiment, a single multimode fiber is used for both light delivery and collection. Thus, a single multimode fiber may be used as a lens-less, in vivo imaging device with a sub-micrometer resolution. In some embodiments, a postprocessing technique may be employed to compensate for modal scrambling inherent with multimode fibers. For example, light focused into a multimode fiber may be coupled in different modes, which propagate with different propagation constants and interact by coupling energy from one mode into another. This modal scrambling may lead to partial or complete distortion of an input image at the output of the fiber. However, by employing certain post-processing techniques, the distorted output may be recovered.

The photometric imaging sensor array may be fitted with light spectrum resolving elements, such as, for example, a spectral filter array or a diffraction grating. The use of a spectral filter array or diffraction grating may enable resolution of light intensities received from the image field into constituting spectra. For example, a spectral filter may be used to either select or eliminate information from light based on the wavelength. In some embodiments, this is effected by passing light through a glass or plastic window that has been specially treated to transmit or absorb/reflect some wavelengths.

The light source, which is useful to illuminate a tissue, may comprise a multiwavelength or multispectral array of lights that can be illuminated one at a time to excite the tissue by light of a certain wavelength. This can be composed of, for example, multiple laser diodes or light emitting diodes multiplexed in a single or multi package light source assembly. Alternatively, the light source may be configured to emit pluses of broad spectral white light.

Treatment of certain medical conditions requires frequent intravascular access. For example, cancer treatment often involves frequent access to a subject's central venous system to deliver chemotherapy agents. Unfortunately, repeated needle insertions into blood vessels of a subject can lead to narrowing or collapse of the blood vessels. To avoid these unwanted side effects, implantable port-catheters may be used to provide long term direct access to a subject's central venous system.

Accordingly, some multi-spectral imaging devices of the invention may include a chemotherapy access port that includes a plurality of sensors integrated with, operably or communicatively coupled to, and/or otherwise connected to the chemotherapy access port. The sensors may be part of a sensor assembly, which may be embodied as a system on a chip such as, for example, field-programmable gate array, an application-specific integrated circuit, and/or another programmable hardware device. Various configurations of the sensor assembly are described in further detail below.

Preferably, the access port includes a reservoir 121 covered by a self-sealing septum for receiving fluids (e.g., chemotherapy agents).

Devices of the invention preferably include one or more power modules 107. The power module 107 may include several components, including a power manager, a battery, and a charging circuit. The power manager may be configured to manage and maintain the power supply that the battery supplies for the various components of the device including the distal sensor assembly.

In at least one embodiment, devices of the invention include a sensor interface module that is configured to communicate with various physiological sensors that are integrated into the device. In such an embodiment, the sensor interface module may be configured to communicate one or more physiological indicators to a computing device, e.g., a central server via a data network, a health care professional's device, a local computing device. In some embodiments, the sensor interface includes a power bus that is included with the sensor connection that supplies power to the sensor micro-assembly, as well as a data bus included in the sensor connection for communicating data between the sensor micro-assembly and the sensor interface. In some embodiments, this interface may be configured to use the inter-integrated circuit (“I2C”) protocol which is a half-duplex bidirectional two-wire bus system for serial communication between different devices, or a Serial Port Interface (SPI) bus protocol which is a higher speed bidirectional communication bus between integrated circuits

Other physiological sensors may be integrated at the tip, or near the tip, of the cannula. For example, in some embodiments a fiber optic-based pressure sensor or a fiber optic-based temperature sensor may be incorporated into the cannula. In some embodiments, a multimodal fiber having a Fiber Bragg grating may be etched within the cannula. The Fiber Bragg grating may comprise a short segment of optical fiber that reflects particular wavelengths of light and transmits all others. In some embodiments, this is achieved by creating a periodic variation in the refractive index of the fiber core, which generates a wavelength-specific dielectric mirror. Accordingly, a fiber Bragg grating may be used as an inline optical filter to block certain undesired wavelengths or may be used as wavelength-specific reflector. The Fiber Bragg grating may be coupled to other reactive elements to, for example, translate instantaneous changes in pressure or temperature to a spectral pattern that corresponds to such change in pressure, temperature, or both simultaneously.

In some instances, devices and methods of the invention may rely on speckle tracking photometry to detect and analyze one or more blood analytes. Speckle tracking photometry may relate to a flow-imaging technique combining high-frame rate imaging capabilities of the invention with image pattern matching (speckle-tracking) to directly measure and visualize one or more blood analytes vector velocity fields. For example, devices and methods of the invention may employ speckle tracking photometry to analyze motion of tissues (e.g., blood) in the blood vessels by using the naturally occurring speckle patterns of one or more analytes flowing inside blood. Visualization of the blood speckle movement generally requires high frame rates (in the kilohertz range), which are achieved with devices of the invention. The patterns may be random and analyte specific. Accordingly, some analyte may have a unique speckle pattern (also called patterns, features, or fingerprints) that allows one or more analytes to be detected.

The sensing end (i.e., distal end) of the cannula may be designed according to any of at least three configurations described below. The distal end may comprise an axial sensing configuration, a circumferential sensing configuration, or, more preferably, a third configuration that combines both configurations to enable all around field of view around the cannula tip and in front of the cannula tip.

Utilization of an implanted port-catheter device, as provided by this disclosure, for in vivo assessments can enable active measurements at clinically-appropriate interval reporting time points (e.g., hourly, daily, weekly, etc.) as determined by a healthcare professional for early detection of blood count aberrations, such as, anemia, neutropenia, leukocytosis or thrombocytopenia, which can indicate increased risk for infection, inflammation, or bleeding. Furthermore, port-catheter devices of this disclosure also provide for blood flow rate and velocity assessments, which can enable longitudinal measurements at reporting intervals that are clinically appropriate for early detection of heart aberrations, for example, as measured by ejection fraction and cardiac output parameters.

As discussed herein, in some embodiments, devices of the invention leverage intrinsic characteristics of blood cells to define blood cell types. Devices of the invention may combine assessments of autofluorescence with spectral dynamic imaging to enable enhanced identification of blood cells. Specifically, photosensors may be configured to detect, record, and quantify autofluorescence and specific light absorption and scattering properties based on cell type in response to excitation waves to obtain average red blood cell, white blood cell, and hemodynamic parameters.

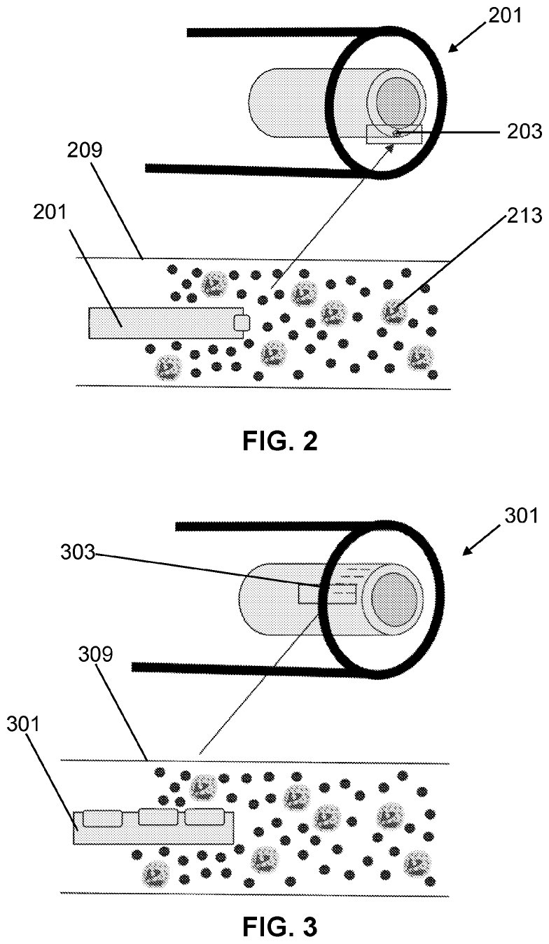

FIG. 2 shows a device 201 with a multi-spectral imaging sensor 203 in an axial configuration. As illustrated, the device 201 is dimensioned for implantation within a blood vessel 209 of a subject. An enlarged view of the device 201 is illustrated in the panel above the blood vessel 209. The multi-spectral imaging sensor 203 comprises an axial configuration. The axial configuration provides a large core fiber optic useful for detecting analyte 213 flowing towards or away from the device 201 within the blood vessel 209.

The axial sensing configuration may comprise a large core multimodal fiber optic. The large core multi-fiber optic may comprise a plurality of cores, e.g., small cores ˜5-10 um, medium Cores ˜25-50 um, large Cores ˜100-250 um, and may employ low NA (Numerical Aperture) vs high NA cores to widen or narrow the field of view of the sensor according to the intended application or the size of the blood vessel in which the sensor is implanted.

FIG. 3 shows a device 301 with multi-spectral imaging sensors 303 arranged in a circumferential configuration. In particular, FIG. 3 shows a distal portion of the device 301, such as a port-catheter, positioned inside a blood vessel 309. The circumferential configuration (also referred to as a tangential configuration) provides multi-spectral imaging sensors 303 (depicted as dashed lines) around a circumference of the distal end. The circumferential configuration may be useful for detecting one or more analytes as the analytes flow around a tip of the cannula.

Preferably, the sensors are arranged in an array format. For example, the sensors may be placed at predefined distances from one another. The predefined distances, and time it takes for the light to be detected by at least two multi-spectral sensors, may be used to calculate various properties of the bloodstream, such as, for example, a blood flow rate and velocity, an ejection time, an ejection fraction, cardiac output, and/or combinations thereof, during different phases of the cardiac cycle regardless of the intensity of such fluorescence or the pattern of change in such autofluorescence intensity. This allows for simultaneous measurement of the flow rate and velocity of blood and counting and classifying one or more analytes as the analytes pass the tangential sensor assembly.

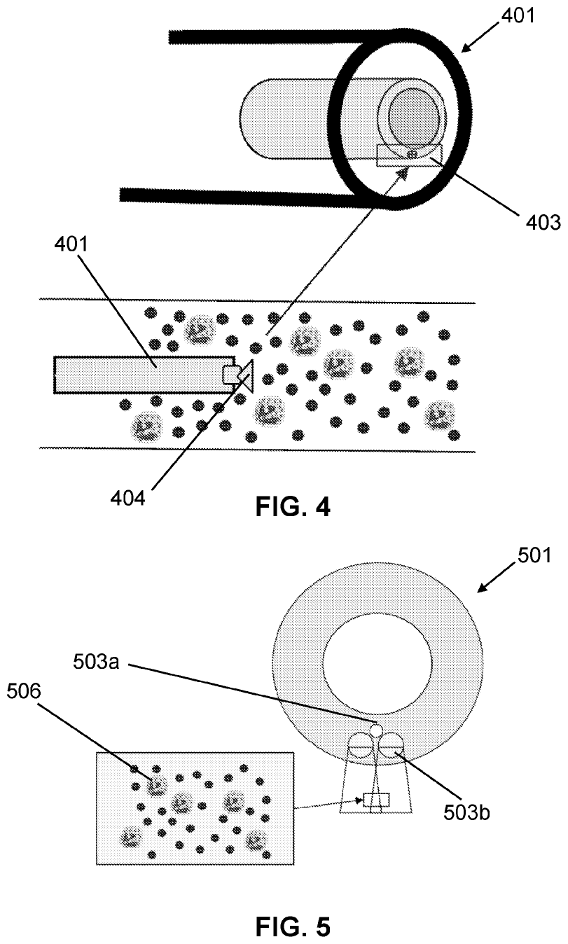

FIG. 4 shows a device 401 with a multi-spectral imaging sensor 403 equipped with a cone mirror 404. The cone mirror 404 may be useful to facilitate capture of light reflected towards the device 401 by one or more analytes by providing a mechanism for funneling reflected light, within a blood vessel, towards the sensor. Accordingly, the cone mirror 404 may, advantageously, increase an area of the sensor configured for detection of one or more analytes within blood vessel by maximizing the area of the device 401 capable of receiving reflected light.

FIG. 5 shows a device 501 with multi-spectral sensors 503 arranged in an axial configuration 503a and a circumferential configuration 503b. The combined configurations may be useful to enable all around field of view of analyte 506 flowing inside a blood vessel.

The device may comprise a single multimodal large core fiber optic with high NA (>0.5). The single fiber optic is preferably 250 micron-500 microns. The device may further comprise a fiber optic bundles with small core fibers of low NA (<0.2). The fiber optic bundle may be 0.35 to 1.0 mm, 5-20 micrometers per fiber. Illumination/excitation capacity of each fiber may be 400 nm-2000 nm.

In some embodiments, the multi-spectral sensors may comprise a Gradient-index (GRIN) lens. GRIN relates to a branch of optics covering optical effects produced by a gradient of the refractive index of a material. Such gradual variation can be used to produce lenses with flat surfaces, or lenses that do not have the aberrations typical of traditional spherical lenses. Gradient-index lenses may have a refraction gradient that is spherical, axial, or radial.

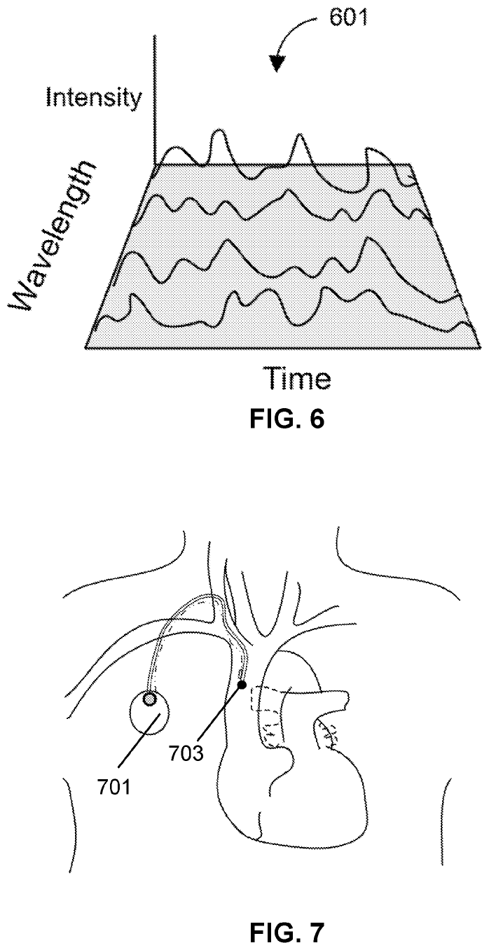

FIG. 6 shows an exemplary output graph produced by devices of the invention. In particular, illustrated, is a plot of at least four distinct wavelengths (e.g., wavelengths between 400-900 nanometers) with intensity values sensed over time by a multi-spectral imaging sensor. The values may correspond to measurements collected from one or more different analytes in blood. The measurements may be plotted as intensity over time to produce signatures indicative of parameters of blood. The measurements may be analyzed by a computer algorithm, such as a machine learning algorithm, to classify the one or more analytes based on temporal changes in multispectral photometric data. For example, a machine learning algorithm may be trained to recognize and identify different classes of white blood cells in a background of surrounding red blood cells. Accordingly, blood cell counting may be performed, and a histogram may be generated to visually represent differential blood cell counts averaged over periods of time spanning a few minutes to a few hours, or longer.

Certain aspects of the invention may employ algorithms, such as machine learning algorithms, to differentiate and assess blood cell subtypes. Of note, Applicant has recently found that unlabeled blood cell, including red blood cells (RBCs) and white blood cells (WBCs), can be identified and differentiated, using an algorithm, by their inherent autofluorescence intrinsic features. These findings were observed using a coherent fiber optic bundle and a flow slide connected to an inline silicone tubing. EDTA (chelating agent) collected blood was circulated through silicone tubing and image sequences were recorded at high frame rates up to 60 frames per second to thereby capture images of blood cells flowing through the flow slide. The coherent fiber optic bundle was connected via an adaptor to a 2-megapixel, complementary metal oxide semiconductor (CMOS) sensor that captured images at the rate of 60 Hz. Applicant observed that white blood cells (WBCs) exhibit higher levels of photo emissions than red blood cells (RBCs) and preferentially flow marginally to the red blood cells stream. Applicant further identified that white blood cells were surrounded by a hollow zone that separated them from the surrounding red blood cells. These identifiable features can be leveraged via devices and algorithms of the invention to segment and track white blood cells (or other types of blood cells) as they flow past optical autofluorescence imaging sensors and/or multispectral sensors. An image processing algorithm has previously performed segmentation of pixels (blobs) with high white blood cells score from pixels with a high red blood cells score. The score was an arbitrary parameter indicating the probability of a pixel representing a RBC population or to WBC population. The algorithm was implemented with an empirical deterministic feature list that included blob intensity, size, and the presence of a nuclear pattern inside the blob representing the blood cell. The algorithm was followed by a visualization routine that overlayed red ellipses over the blobs identified as red blood cells and black outlines over the blobs that were identified as nucleated white blood cells. Accordingly, the algorithm was useful to differentiate white blood cells and red blood cells, and, via systems of the invention, has the potential to classify white blood cells (or other types of cells) to differentiate among subtypes.

For example, to identify and differentiate white blood cells and red blood cells and classifications thereof into differential subtypes for health evaluations, devices of the invention may integrate a machine learning algorithm that utilizes a convoluted neural network to perform the segmentation and recognition of red blood cells and white blood cells to thereby provide the ability to differentially classify white blood cells into its subtypes, such as, neutrophils, basophils, eosinophils, lymphocytes, and monocytes. In certain instances, an additional computational pipeline for blood cell counting and histogram generation routines may be employed to run simultaneously in parallel with the image processing pipeline to generate an analytical report of the differential blood cell counts to provide helpful clinical data.

Accordingly, some aspects of the invention may include the use of machine learning systems (machine learning algorithms) to automatically preform analyses through experience and by use of data. Such machine learning algorithms may build a model based on sample data, known as training data, in order to make predictions or decisions without being explicitly programmed to do so. The training data may include plots of different wavelengths (e.g., wavelengths between 400-900 nanometers) with intensity values plotted over time. The plots may be associated with known patient statuses, such as, low blood counts, neutropenia, or anemia. Using the training data, machine learning systems may learn to identify features of multi-spectral imaging data, such that, when the machine learning system is presented with new data (i.e., patient data) the machine learning system is operable to identify features related to a health status based on correlations with the training data.

Preferably, such machine learning systems employ one or more convolutional neural networks (CNNs). CNNs are a class of deep neural networks useful for analyzing visual imagery. The CNNs may consists of an input layer, hidden layers and an output layer. The hidden layers may include layers that perform convolutions. This may include a layer that does multiplication or other dot product. The CNNs may further use other layers, such as pooling layers, fully connected layers, and normalization layers.

According to some embodiments, devices of the invention are useful to classify and distinguish RBCs from WBCs (or their respective subtypes) using a combination of autofluorescence and multi-spectral profiling of the cells. For example, a fiber optic sensor system emitting light covering a broad spectra of wavelengths, e.g., from 405 nm-850 nm, which encompasses autofluorescence and spectral imaging over the visible to infrared range of light, may be employed to excite cells within a subject. Reflected scattered light can be captured via a camera sensor fitted with a wavelength-resolving component (e.g., a holographic grating). In some instances, the system will combine and iterate supervised and unsupervised training of a Convoluted Neural Network (CNN) algorithm to distinguish between blood cell types repeatably and enable an average count over time. Cells can be, for example, distinguished by a trained observer to extract the data to be used in supervised training of the algorithm. In addition, the algorithm can be trained to distinguish cell types using unsupervised training strategies, in which, for example, cells are labelled with fluorescent markers, one population at a time. The training algorithm may use fluorescent labelling of a population of interest in a given sample as an identifier of the class or type of the blood cells and leverage the information to take other characteristics of the observed cells as inputs in the training algorithm. Advantageously, systems of the invention are useful to perform high resolution/high sampling rate multispectral imaging and classification of blood cells passing or flowing by the field of view of the imaging sensor at a core of a blood stream at high velocities, for example, such as velocities higher than in skin or mesenteric capillaries.

In some embodiments, the signatures may be recorded to a memory unit of the device. The signatures may be analyzed by, for example, correlating the recorded signatures to one or more patients having a known heath status. Correlating may involve aligning the signatures to signatures associated with a known health status (e.g., neutropenia, anemia, sickle cell disease) and determining an alignment score. An alignment score above a pre-determined threshold (e.g., between 0.7 and 1.0) may indicate a positive association (i.e., that the subject identifies with the known health status). The signatures may be correlated against signatures from a plurality of patients having a known health status (e.g., a healthy or diseased status) to assess the subject's health status. Accordingly, signatures produced by devices of the invention may be useful to manage patient treatments by quickly identifying whether a particular treatment is effective.

Moreover, other measurements may be plotted over time, for example, red blood cell count, white blood cell count, or platelet count. In some embodiments, measurements are plotted by a plotting module associated with the device. The plot module may be generated by a processor within the implantable device or may be provided by a computing device external to the subject.

Measurements made by the devices of the invention may be communicated to one or more external devices via a communications module. Preferably, the communication module is integrated within a chemotherapy access port of the device. The measurements may relate to physiological indicators of treatment or disease which are indicative of a change in health status.

Preferably, measurements taken by devices of the invention are transmitted to a local computing device. The transmission may occur by way of, for example, Bluetooth. In some embodiments, devices and methods of the invention transmit physiological data to a central server. For example, in one embodiment, if the method determines that blood parameters are within a predefined/expected range then the method continues to measure one or more physiological condition indicators/parameters, e.g., indicators of infection, disease, or a chemotherapy-related physiological condition, using one or more sensors integrated with the device. Otherwise, the method, in one embodiment, generates and sends healthcare alert messages to an authorized user such as a healthcare provider.

In another aspect, this disclosure provides methods for monitoring patient health using implantable multi-spectral sensors. Methods of the invention may be useful to monitor changes in health status of chronically ill patients. For example, methods of the invention may be useful to identify when a chronically ill patient needs a treatment, and how urgently the treatment is needed. For example, such as a growth factor treatment, or a steroid treatment, in conjunction with chemotherapy.

Methods provide for signal acquisition and analysis of multi-spectral patient data. Methods may generally include the steps of emitting light via a light guide or fiber optic filament or bundle or strand onto blood cells flowing in a subject's bloodstream (the sample) from a broad-spectral light source (e.g., a light emitting diode) or from a multiplexed multispectral light source. Light reflected from the cells located at the sensor's field of interest or field of view (FoV) may be relayed through a fiber optic bundle or the single multimodal fiber to a photometric sensor array passing through different optical elements on its way such as lenses, mirrors, diffraction grating, optical filters, or the like. Light patterns that arrive at the photosensor array may be converted to digital representation of the light intensities that are transferred to the microprocessor for analysis. A microprocessor may perform a series of signal and image processing operations in the image domain, time domain, spectral domain, and wavelet domain including but not limited to normalization, deconvolution, thresholding”, edge finding, blob segmentation, statistical analysis of size and multispectral intensity, Fourier transform and short time Fourier transform (STFT), coherence analysis, continuous wavelet transform, and wavelet scattering transform

Methods of the invention may include implanting a device into a subject. The device preferably includes a multi-spectral imaging sensor system configured to sense one or more analytes (e.g., a blood cell, a circulating tumor cell, a protein, a microbe or a nucleic acid etc.) in the subject. Sensing of the one or more analytes by the sensor may be used to assess a health status, preferably remotely, of the subject. Assessing a health status of the subject may involve correlating data, e.g., intensity values plotted over time for one or more wavelengths, with corresponding data taken from a healthy patient and/or a diseased patient.

FIG. 7 shows a device 701 implanted within a subject. The device 701 includes a multi-spectral imaging sensor system 703 placed directly in a bloodstream via a catheter for measuring various analytes in the blood as it flows or circulates in the bloodstream, for example, through the superior vena cava. Measurements are recorded from the blood and preferably transmitted, e.g., via a communication module, to one or more computing devices external to the subject. For example, the sensors may be configured to detect and measure various circulating elements in the bloodstream such as red blood cells, platelets, white blood cells, and/or other circulating components such as, for example, DNA, proteins, cancer cells.

The device 701 may include a plurality of sensors configured to determine one or more physiological conditions of a subject, for example chemotherapy-related conditions. The one or more physiological conditions may include parameters selected from a red blood cell count, white blood cell count, platelets, and/or blood flow rate and velocity and derived ejection fraction.

Regular monitoring of physiological parameters is important in chronically ill patients, particularly cancer patients who are receiving cytotoxic or immunomodulating therapies and who are potentially immunosuppressed. Additionally, monitoring a patient's physiological responses (e.g. body temperature, heart rate and variability, signs of infection, sleep, oxygen levels, glucose, and cortisol, etc.) before and after therapy infusion would be a desirable method to determine if the patient is benefiting from the current treatment strategy. Current methods for monitoring a patient's physiological parameters primarily focus on the use of external measurement devices such as thermometers (oral, rectal, axillary, ear, or temporal), electrocardiograms (ECG), blood pressure cuffs, and laboratory-based blood analysis. The device will alert physicians if laboratory-based blood analysis is necessary prior to next scheduled treatment.

While these methods are usually effective in hospital settings due to continuous professional healthcare compliance, it may be critical that these parameters are accurately monitored in both inpatient and outpatient settings. Furthermore, surveys from oncologists have suggested that more frequent monitoring of a patient's physiological parameters in both inpatient and outpatient settings may allow healthcare providers to detect adverse effects sooner and potentially avoid further complications from treatment in their cancer patients.

To address this need, the invention herein provides an implantable venous access port device which contains a plurality of sensors including multispectral sensors for longitudinal and/or interval physiological parameter monitoring and an optionally embedded microprocessor which may be configurable to collect, analyze, store, and transmit the physiological data over standard computer networks. Optionally, in any embodiment, the processor may be configured either onboard or separately from the catheter device. The microprocessor may contain a wireless transmitter that may also be capable of transmitting the stored physiological data via encrypted wireless communication links to a secure local computing device. The local computing device will transmit the physiological data to a central server via encrypted wireless communication links. Healthcare providers and patients may securely access patient data from the server using a designated platform upon subject authentication. For example, as described in co-owned Pat. Pub. No. US20210016074A1, which is incorporated by reference.

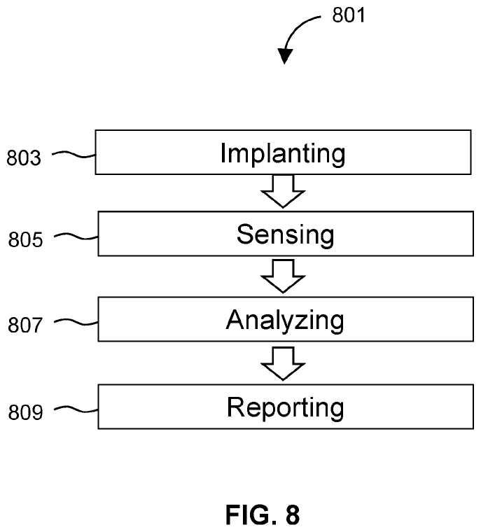

FIG. 8 shows a method 801 for remote monitoring of patient health. The method involves implanting 803 a device into a subject. The device, as described in FIG. 1, includes a multi-spectral imaging sensor system configured to sense an analyte in the subject. The method further includes sensing 805 of the analyte by the sensor to generate data useful for assessing a health status of the subject. Assessing a health status may involve analyzing 807 the data generated by the sensing step. For example, analyzing 807 may involve correlating signature profiles with signature profiles taken from a subject with a known health status. Finally, methods of the invention involve reporting 809 a health assessment to a treatment facility or the patient.

In at least one embodiment, the subject is animal, wherein the animal is a pet, a non-human primate, a research animal or a large animal. For example, the subject may be one of a cat or a dog. Accordingly, in some embodiments, methods and devices of the invention are useful for application in Veterinary Medicine.

In certain embodiments, the remote monitoring system may be configured to send patient health alerts to one or more healthcare providers, such as, the subject's treating physician. The alert may be provided when a physiological parameter deviates from a pre-determined threshold. The thresholds are configurable to be set by the healthcare provider using a designated platform. It may be noted by one of skill in the art that research and other disease interventions show that remote patient monitoring be beneficial to patients. Thus, the devices and methods described herein provide opportunities not found in existing systems to monitor physiological parameters of a subject. When these parameters deviate from the set thresholds for a designated period of time, alert messages are generated by the platform and sent to the healthcare providers via wireless communication links (e.g. email or text message). In various embodiments, the physiological parameters to be monitored may be selected by the healthcare provider and the thresholds of what constitutes a threshold sufficient to trigger an alert may be based on research and/or clinical norms in the relevant field. In some embodiments, data from the apparatus, system, and methods disclosed herein may provide data that upon proper authorization may be entered into a patient's electronic medical records (“EMR”).

For example, according to certain embodiments of the invention a health status, or an alert, may be transmitted, via a communication module, from the device to a computing device external to the subject. The transmission of data may occur via Bluetooth radio technologies. The computing device may be operable to provide the health status or alert to a treatment facility via a data network. The data network may be a wireless data network. For example, such as, include Wi-Fi wireless data communication technology, a personal area network (PAN), a local area network (LAN), a wireless local area network (WLAN), a campus area network (CAN), a metropolitan area network (MAN), a wide area network (WAN), a storage-area network (SAN), a system-area network (also known as SAN), a passive optical local area network (POLAN), an enterprise private network (EPN), a virtual private network (VPN). digital subscriber link networks (DSL), various second generation (2G), third generation (3G), fourth generation (4G), fifth-generation (5G) cellular-based data communication technologies.

According to some aspects, methods and devices of the invention rely on optical fibers for multi-spectral imaging of one or more blood analytes. Optical fibers are ideally suited for imaging deep into the body, e.g., a blood steam, at high resolution, where scattering makes standoff imaging impractical. Owing to their small size, optical fibers can be inserted directly into regions such as blood vessels. Since optical fibers are thin, tissue damage can be minimal, and typical wavelengths of operation in the visible and infrared are non-ionizing.

Despite these features, one fundamental physical problem stands out for fiber-optic imaging: how to transmit an optical image through an optical fiber. Optical fibers scramble spatial information. If one attempts to relay a spatially and temporally coherent image (from a laser) through an optical fiber, the output will be a speckle pattern. This may be a result of a wide variety of physical phenomena inside the fiber, from modal coupling to geometrical and material imperfections in the fiber.

The ratio between fiber optic image bundle and the imaging sensor array enables analyzing light modulation patterns induced by blood cells flowing in the bloodstream. Table 1 identifies useful sensor to fiber ratios for performing methods of the invention.

TABLE 1
Sensor-to-Fiber Ratio

| Level | Ratio | Notes |
|---|---|---|
| Level 1 | 9:1 | Minimum |
| Level 2 | 25:1 | e.g. 10,000 fiber cores require |
| | | 250,000 pixels |
| Level 3 | 49:1 | e.g. 10,000 fiber cores require |
| | | 490,000 pixels |
| Level 4 | 64:1 | |
| Level 5 | 81:1 | |
| Level 6 | 100:1 | |

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. Circuitry may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry. Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Certain aspects of the invention may relate to principles of electrical impedance cytometry. is a technique whereby the dielectric or impedance properties of biological cells are measured. An externally applied electric field is used to probe the cell or sample of cells. This can be achieved either through the application of one or more discrete excitation frequencies or via broadband frequency measurement techniques. Typically, a potential is applied between a pair of electrodes and the resulting current flowing through the system is measured. The impedance of the system is the ratio of the voltage to the current passing through the system. The dielectric properties of the cells can be derived from this measurement through the use of appropriate models. The development of microfluidics and lab-on-a-chip type devices has allowed single cell impedance measurements to be performed with high sensitivity and high throughput.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### EXAMPLES

### Example 1—Implantable Catheters for Assessing Health in Pediatric Patients

Implantable venous access devices (IVAD's) either centrally or peripherally implanted, are used to assist treatment in children with a variety of diseases. These diseases include neoplasms, hemophilia, long-term supplement needs and metabolic/endocrine diseases, as examples. In the area of neoplasms, approximately 85% of pediatric cancer patients have a long-term chemo-port catheter in use to facilitate drug infusions and blood sampling. Totally implantable port-catheters are preferred in children with solid and hematological malignancies because of decreased pain related to injections, the rate of infection, and ability to maintain patency for the long term. Despite widespread use, Complications in central venous access devices have been reported to be as high as 40%. Pediatric patients receiving conventional chemotherapy for cancer have an increased risk of infections, and represent acute life-threatening events in these immunocompromised patients. Pediatric patients with infections are normally immediately hospitalized and treated with IV antibiotics. Symptom monitoring in pediatric patients through use of patient-reported outcomes (PROs) is uncommon due to difficulty obtaining accurate and consistent information from children experiencing complications. This invention describes the ability to monitor pediatric patients, remotely and passively, for complications by measuring physiologic functions through an implantable port-catheter or other implanted intravenous access devices equipped with optical sensor technology.

In certain embodiments, implantable devices (e.g, ports) with remote monitoring capabilities are provided for assessing health of a patient with an implanted chemo-port or another implanted intravenous access device (IVAD). The implantable devices can include one or more optical sensors that enable detection of one or more analytes inside the subject's body, using photonic means such as autofluorescence, multi-spectral (MSI) and hyperspectral imaging (HSI), covering the wavelengths from 400-2000 nm. For example, in preferred embodiments, the devices may relate to ports with one or more optical sensors capable of remotely measuring symptoms of physiological distress (e.g., fever, heart rate aberrations, blood cell count fluctuations, blood flow velocity and blood flow rate) via the spectral sensors. In some embodiments, the ports can be used to deliver chemotherapy treatments and monitor patient health over the course of chemotherapy treatments. In some embodiments, IVAD's may be utilized in non-cancer disease states, such as endocrine, cardiovascular and autoimmune diseases, and can also be equipped with optical sensors to monitor patient health status.

Spectral sensors can sense physical light properties modulated (e.g., reflected, absorbed, dispersed, scattered, etc.) by one or more analytes present in the body to allow for earlier detection of signs indicative of a health status. Identification and count of prokaryotic cells, including blood cells, tumor cells, fibroblasts. etc. and molecular analytes can be further characterized by use of spatial profiling via HSI. Use of photonic sensing technology can also be applied to detection of eukaryotic cells such as fungi and bacteria. This is useful for, among other things, early detection of complications associated with implantable ports, e.g., infections or thrombosis, and to evaluate patients prior to a scheduled chemotherapy treatment. Early detection is especially critical for pediatric patients who may not have the ability to describe symptoms of complications, and frequently are hospitalized over the course of cancer treatment or other types of treatment associated with chronic or acute diseases in children.

Potential advantages of using IVAD's to deliver drugs and sample body fluids can include, for example:
1) Reducing the number of needles sticks for medication, fluids, nutrition, blood products and blood samples.
2) Reducing the irritation of veins exposed to drugs.
3) Ability to infuse multiple types of medication at the same time.
4) Children can continue activities.
5) Ensuring complications are identified and addressed quickly.

Vascular access devices in children can include arterial, venous and intraosseous, and cover indications ranging from emergency, urgent and elective.

The type of IVAD's depends on the access site and include the following:

Peripheral venous, midline access, peripheral inserted central catheter (PICC), non-tunneled central catheter, tunneled central venous access (Hickman's/Broviac or similar), implantable port access, intra-osseous access, arterial access, umbilical (arterial and venous). IVAD's can be used to infuse cancer therapy and adjuvant therapies, provide liquid nutrition, and infuse antibiotics and anti-fungal medicines in the case of infection.

Catheter sizes may depend on type and age of pediatric patient with ranges of 20-26 G and 1-7 French. Dwell time can also vary depending on the type of IVAD, which can range from days (short-term), up to 6 months (intermediate-term) or 6 months or longer (long-term). As the length of the catheter is typically sized to the individual, devices of the invention may be embodied in one of the following manners: a) the sensors may be molded into the catheter such that the catheter may be cut to size without damage to the sensors, e.g., where the sensors are located in a clinically relevant location but distal to the catheter tip, b) the device can have various pre-sized catheter lengths, or c) the device can have a dual or triple lumen catheter where the sensor bundle will be threaded through the secondary lumen to desired length allowing for shortening and attachment at the port. The implant time may vary depending on the treatment, which can range from days (short-term), up to 6 months (intermediate-term) or 6 months or longer (long-term).

As described in U.S. patent application Ser. No. 16/932,503, incorporated herein by reference and described above, the devices of the invention may be equipped with wireless communication components that provide for remote transmission of clinically actionable data to one or more locations, such as, one or more treatment facilities. Devices of the invention, for use in pediatric patients, may further include any of the features described more generally above.

In some embodiments, the assembly of these components may be designed into the “port” of a chemo-port which is implanted subcutaneously in the subject. In some embodiments, the port may not be implanted subcutaneously, but rather attached to the end of one of the catheter lumens accessible outside the body, and held into place with surgical tape, arm band, chest band or similar.

Data collected previously, and in the pediatric population described herein, may include, for example, information related to one or more of red blood cells, white blood cells, platelets, proteins, electrolytes, circulating tumor cells, microbes, nucleic acids and/or hemodynamics, and cardiac function, including blood flow rate and velocity and cardiac output, oxygen level, heart rate, body temperature, blood pressure, and ECG measurements of the patient.

As previously described in U.S. patent application Ser. No. 16/932,503 and as discussed above, certain aspects of the invention may be used to address a long-standing problem in the healthcare industry, which is that throughout patient treatment, the clinical status of a patient (including pediatric patients) is largely unknown.

Additionally, devices of the invention are useful to address problems associated with the rising volume of emergency room (ER) visits for pediatric cancer treatment-related toxicities when new or worsening symptoms emerge in between clinic visits. Such problems are well documented. For example, one study in pediatric cancer patients showed that the two most common diagnoses in the ER are fever and fever with neutropenia. Of the patients studied, 44% were admitted to the hospital, and those with febrile neutropenia were admitted at a rate of 82%. Furthermore, these recognizing these complications is more difficult and delayed in pediatric patients due to diminished communication ability.

Implantable intravascular devices are described above herein having remote physiological monitoring capabilities in adult patients. That same technology may be specifically applied in pediatric patients with implantable IVAD's, including chem-ports, PICC lines and tunneled catheters. Such devices may include multi-spectral and hyperspectral sensors that, when placed in contact with a subject's circulatory system, are capable of sensing one or more analytes to provide clinically actionable data related to the subject's heath status. As noted, any aspects of the devices and methods described above and herein can be applied to pediatric patients including the use of an implanted port-catheter device, as provided by this disclosure, for in vivo assessments that allow for active measurements at clinically-appropriate interval reporting time points (e.g., hourly, daily, weekly, etc.) as determined by a healthcare professional for early detection of blood count aberrations, such as, anemia, neutropenia, leukocytosis or thrombocytopenia, which can indicate increased risk for infection, inflammation, or bleeding or other adverse events associated with investigative drugs or devices.

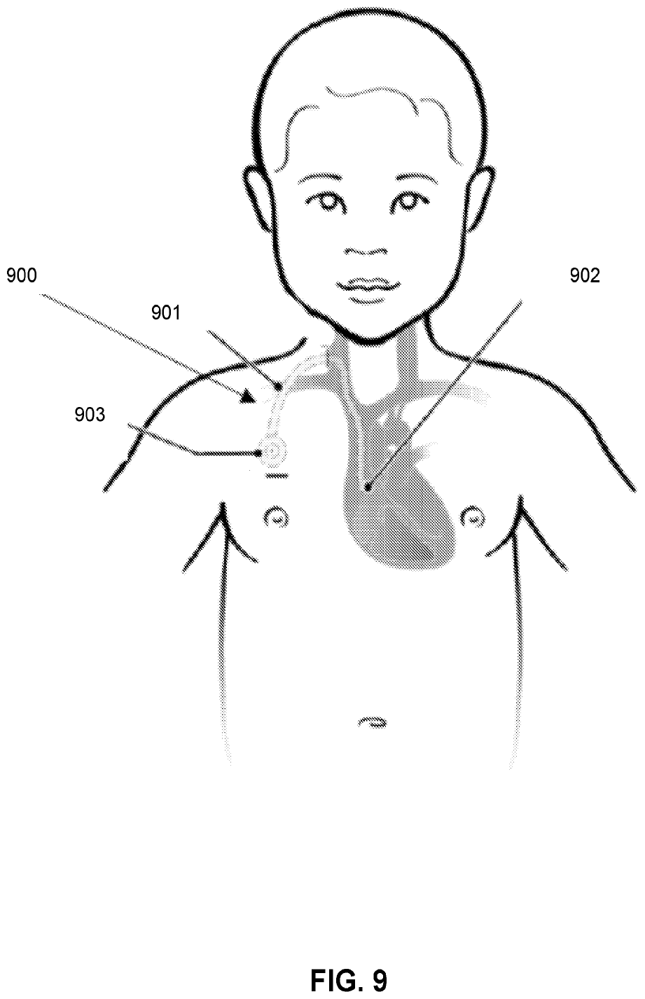

FIG. 9 shows an example a device 900 implanted within a pediatric subject. The device 900 includes a spectral imaging sensor system 901 placed directly in a bloodstream via a catheter for measuring various analytes in the blood as it flows or circulates in the bloodstream, for example, through the superior vena cava 902. Measurements are recorded from the blood and preferably transmitted, e.g., via a communication module, to one or more computing devices external to the subject. For example, the sensors may be configured to detect and measure various circulating elements in the bloodstream such as red blood cells, platelets, white blood cells, and/or other circulating components such as, for example, DNA, proteins, cancer cells.

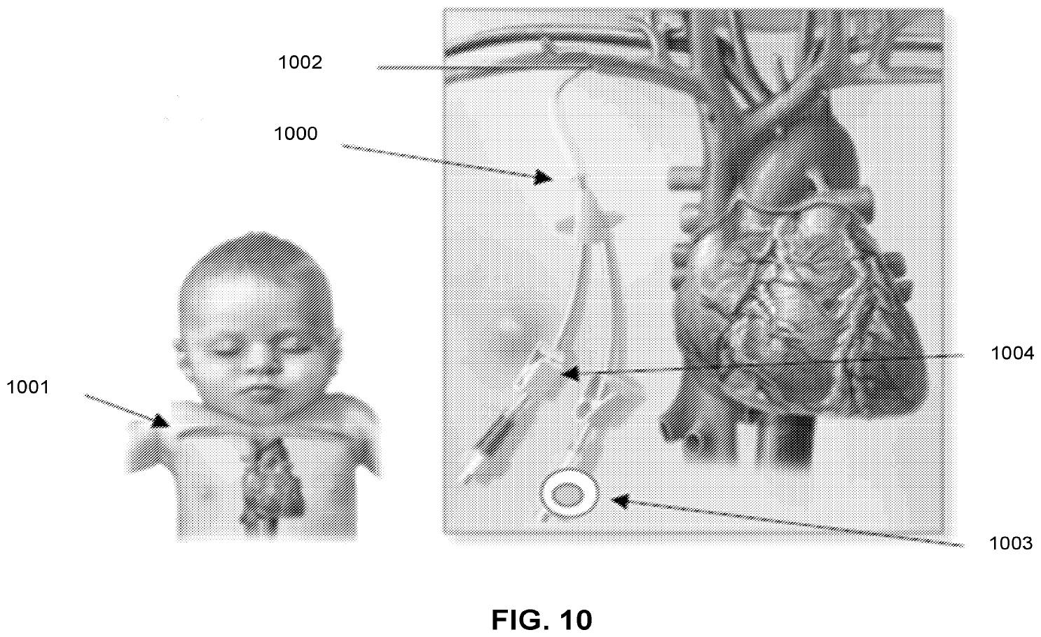

The port of the device 903 may be equipped with components described in U.S. patent application Ser. No. 16/932,503 and can be placed under the skin near a large vein in the upper chest. FIG. 10 depicts a tunneled central venous catheter 1000 implanted in a neonate 1001. In some embodiments, the tunneled central venous catheter is inserted into a central vein 1002. The device 1000 has an external device port 1003 with lumen, that may be equipped with the components such as those described in described in U.S. patent application Ser. No. 16/932,503 and is attached to one end of the double-access catheter. In one embodiment, the loose device port is taped to the skin of the pediatric subject with surgical tape. The catheter may also have clamps 1004 that provide access or close off access to the catheter.

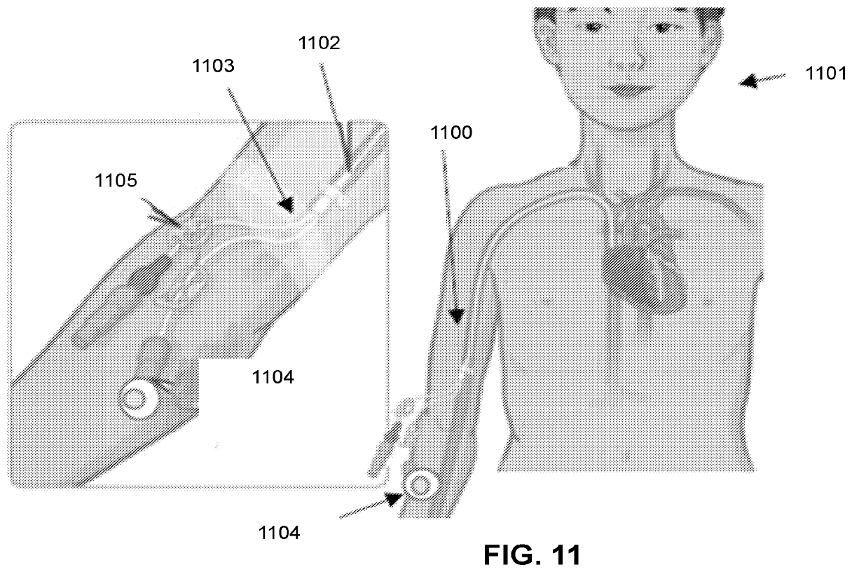

FIG. 11 shows an example of a peripherally inserted central catheter (PICC) line 1100 implanted in a pediatric subject 1101. The catheter 1102 is inserted into the vein of the arm 1103. The device 1100 has an external device port 1104 with lumen, that may be equipped with components described in U.S. patent application Ser. No. 16/932,503 and is attached to one end of the double access catheter. In one embodiment, the loose device port is taped to the skin of the pediatric subject with surgical tape. The catheter may also have clamps 1105 that provide access or close off access to the catheter.

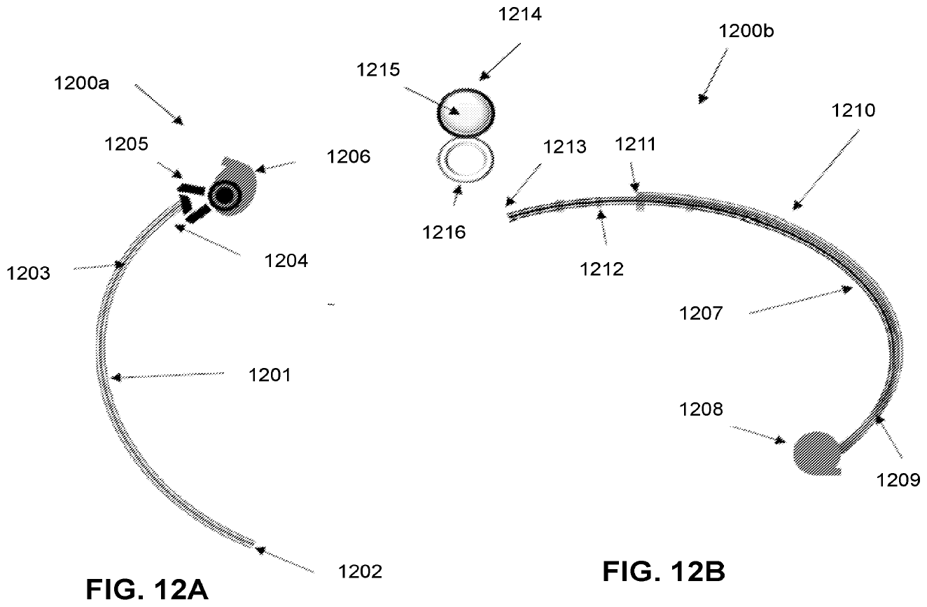

FIGS. 12a and 12b depict embodiments of sensors in the catheter. The sensor bundle is either part of a dual lumen or co-extruded with the catheter. The catheter is often cut to size once the appropriate placement is determined within the animal. Therefore, it is essential to ensure the sensors are not damaged in the implantation process. The sensor is distal to the catheter tip at a length that cannot be harmed when cutting the catheter to size and/or is advanced through the dual lumen catheter. Alternatively, not shown, the catheter with embedded sensors may be configured in multiple lengths for appropriate selection.

FIG. 12a shows a method 1200a for shortening the length of catheters equipped with the disclosed sensor technology. In this embodiment, a dual lumen catheter 1201 with fiber optic sensors are advanced to the tip 1202 after the catheter is cut to size and in place. The sensors 1203 are shortened at the port end 1204 and fits into a sensor lock 1205 that connects the sensor line to the port 1206. The attachment of the sensor bundle 1205 may be a clamp or fitting that allows a snug fit with the body of the port 1206.

FIG. 12b shows an alternate method 1200b for shortening the length of catheters equipped with the disclosed sensor technology. In this embodiment a catheter 1207 has a port 1208 with an extruded lumen in which the fiber optic sensors 1210 are embedded distal to the tip of the catheter 1213 but within a clinically relevant distance from the tip of the catheter. Specifically, the fiber optic sensors 1210 run a shorter length through the extruded lumen of the catheter, stopping at a location 1211 relevant to measure blood. The catheter has markers 1212 for cutting the catheter to the appropriate length, without affecting the sensor fibers. The tip of catheter view 1214 is shown with the fiber optic sensors 1215 occupying the extruded lumen and is shorter than the length of the catheter tip 1216.

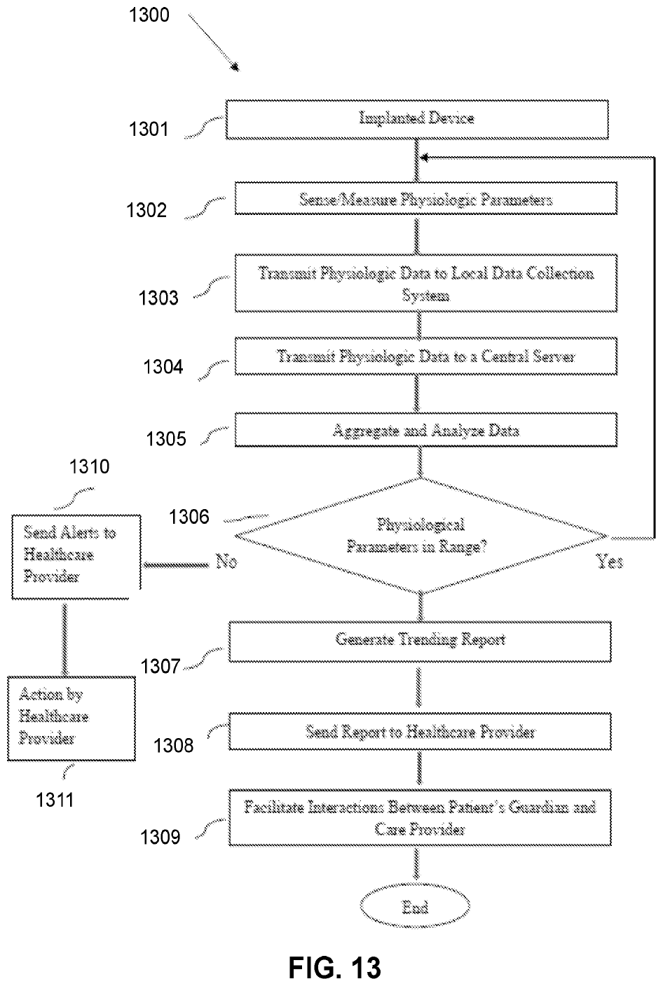

FIG. 13 shows a method 1300 for remote monitoring of pediatric patient health. The device, such as those described above, can include a spectral imaging sensor system, configured to sense an analyte in the subject, which is implanted in the pediatric patient 1301. The method further includes measuring 1302 of an analyte by the sensor to generate data useful for assessing a health status of the subject. In further embodiments, the method 1300 transmits 1303 the physiological condition indicators/parameters to a local computing device which may also be referred to as a local data collection system. In some embodiments, the method includes 1300 transmitting 1304 the physiological condition indicators/parameters to a central server. Assessing a health status may involve aggregating and analyzing the data 1305 generated by the sensing step. For example, analyzing 1305 may involve correlating signature profiles with signature profiles taken from a subject with a known health status.

The method 1300 depicted in FIG. 13 includes determining 1306 that the physiological condition indicators/parameters are within a predefined/expected range, then the method 1300 includes continuing to measure 1302 one or more physiological condition indicators/parameters using one or more sensors integrated with the IVAP device.

If data is in range, the system may generate a trending report 1307 at intervals determined by the healthcare provider, and send the report 1308 to a treatment facility or the patient's guardian. The report would facilitate interactions between the patient's guardian and healthcare provider 1309. In certain embodiments, the remote monitoring system may be configured to send patient health alerts 1310 to one or more healthcare providers, such as, the subject's treating physician when physiologic parameters deviate from the set thresholds for a designated period of time. Alert messages are generated by the platform and sent to the healthcare providers via wireless communication links (e.g. email or text message) and facilitate action to be taken by the healthcare provider 1311, such as ordering of lab tests, blood transfusions or growth factors, and the method 1300 ends.

In some embodiments, data from the apparatus, system, and methods disclosed herein may provide data that upon proper authorization may be entered into a patient's electronic medical records (“EMR”).

The apparatus, system, and methods described in the various embodiments above provide a number of important improvements over existing methods of monitoring physiological conditions of pediatric patients with IVAD's. For example, among the beneficial aspects of the apparatus, system, and methods described in the present disclosure and previous, are to monitor physiological functions in an integrated fashion and in real time and earlier evidence of physiological deviations to expected levels. This saves the time for the patient, physician, and laboratory time, as well as reducing overall costs to health care system.

Aspects of the invention may include a device with an implanted port-catheter or an implanted catheter with an external port, equipped with an autofluorescence, multi-spectral and hyperspectral imaging sensor system to enable remote pediatric patient monitoring of health status. The device may be a port catheter, fitted for pediatric patients, implanted to enable infusion of a pharmaceutical or withdraw blood. The device can be a PICC line fitted for pediatric patients, implanted to enable infusion of a pharmaceutical or withdraw blood. The device may be a tunneled catheter line fitted for pediatric patients, implanted to enable infusion of a pharmaceutical or withdraw blood, or to administer liquid nutrition.

In certain embodiments, the device may be used to measure an analyte in the pediatric patient by autofluorescence, MSI and HSI technologies. The analyte may be a blood cell count, a circulating tumor cells, a protein, a microbe, electrolyte, chemical, drug or a nucleic acid. The device may measure blood flow rate and blood flow velocity. In some embodiments, the device may measure blood pressure, body temperature, heart rate, heart function through an ECG, oxygen level, and electrolyte concentration from the skin.

In certain embodiments, the sensor system (optical, thermal, or electronic, galvanic, impedance, amperometric) may collect this data immediately upon implanting the device. The data collected by the device can be transmitted to a server or through an Industrial, Scientific and Medical (ISM) Band and analyzed covering 400 MHz to 2.4 GHz radiofrequency spectrum. In some embodiments, data may be shown to the recipients as a trending report. If the analyzed data falls outside pre-specified ranges of normal, an alert may be sent to a health care team.

### Example 2—Implantable Vascular Access Devices for Assessing Health in Patients

Systems and methods of the invention may include uses of an implantable central catheter medical device having one or more spectral imaging sensors for assessing health status remotely in patients with therapies or procedures requiring an implanted intravenous access device (IVAD) including a central venous access device (CVAD), a peripherally inserted central catheter (PICC) or centrally inserted central catheter (CICC), or pleural port, herein called IVAD.

As discussed above, implantable venous access devices (IVADs) either centrally or peripherally implanted, are used to assist treatment in patients with a variety of diseases. Symptom monitoring in patients through use of patient-reported outcomes (PROs) currently requires frequent visits to the clinic for blood draws. This invention describes the ability to monitor patients, remotely and passively, for complications by measuring physiologic functions through an implanted intravenous access devices equipped with optical sensor technology.

IVADs are used in patients with various disease states requiring different therapies over an extended period of time such as delivery of fluids, medications and parenteral nutrients; procedures such as dialysis/apheresis; and/or hemodynamic monitoring. Although there are risks associated with the use of IVADs, such as bloodstream infections and thrombosis, they are of increased importance in treatment of the critically compromised or ill patients such as those under emergency or intensive care, and/or requiring surgery. The implantable devices of this disclosure include one or more optical sensors that enable remote patient monitoring thereby providing early detection of such concerns. Specifically, the optical sensors enable detection of one or more analytes inside the subject's body, using photonic means such as autofluorescence, multi-spectral (MSI) and hyperspectral imaging (HSI), covering the wavelengths from 400-2000 nm. For example, in preferred embodiments, the devices relate to catheters with one or more optical sensors capable of remotely measuring symptoms of physiological distress (e.g., high body temperature, heart rate aberrations, blood cell count fluctuations, blood flow velocity and blood flow rate) via the MSI and HSI sensors. In some embodiments, the IVADs are useful to deliver therapeutic treatments and medications and monitor patient health over the course of treatments. The MSI and HSI sensors sense physical light properties modulated (e.g., reflected, absorbed, dispersed, scattered, etc.) and by one or more analytes present in the body to allow for earlier detection of signs indicative of a health status. Identification and count of prokaryotic cells, including blood cells, tumor cells, fibroblasts, etc.) and molecular analytes are further characterized by use of spatial profiling via HSI. Use of photonic sensing technology can also be applied to detection of eukaryotic cells such as fungi and bacteria. This is useful for, among other things, early detection of complications associated with the implantable device, e.g., infections or thrombosis, and to evaluate patients prior to a scheduled treatment. Early detection is especially critical to ensure complications are addressed in a timely manner.

Advantages of using IVAD's to deliver drugs and sample body fluids can be similar to those discussed above with specific respect to pediatric patients. The type of IVADs depends on the access site and are commonly called central venous access devices (CVADs), centrally inserted central catheters (CICCs) and peripheral inserted central catheters (PICCs), and include the types and uses discussed above with respect to pediatric patients. Catheter sizes depend on type and age of patient with typical ranges 5-18 French, however, the devices of this invention may be incorporated into any size catheter including the pediatric sizes discussed above. Dwell times may be similar to those discussed above with respect to pediatric patients and catheter sizing may be accomplished using similar features and methods.

As described in U.S. patent application Ser. No. 16/932,503 and discussed above, spectral sensors may be used to sense physical light properties modulated (e.g., reflected, absorbed, dispersed, scattered, etc.) by one or more analytes present in the body to allow for earlier detection of signs indicative of a health status or measuring and monitoring an analyte during patient treatment or when undergoing a procedure. In some embodiments, devices of the invention can include an assembly of components for detection, analysis, and transmission of clinically relevant data. Catheter devices may be include any of the features described above and ma include a port that enables removal of fluids in pleural port embodiments or an external port as appropriate. Clinically relevant data, as described above, can be obtained using devices of the invention and may be transmitted to locations such as a physician's office or treatment center. Ports may be held in place with surgical tape or a protective arm band. Sensor data may include identifiable chemicals or drugs for use in clinical pain management applications in certain embodiments. Systems and methods of the invention may find application in pleurisy, monitoring drugs of abuse, pain management, and stem cell research. Evaluations using systems and methods of the invention may offer the first clinically relevant information obtained from a patient in an extended period of time and may uncover signs that may indicate physiological stress or adverse events. Devices and methods of the invention may find use in critically ill patients and can help address rising volumes of emergency room visits among that population. This may be particularly useful as recognizing complications in critically ill patients is often difficult due to their compromised state and providing extended data using systems and methods of the invention can help sort through background noise and identify true complications.

Implantable intravascular devices having remote physiological monitoring capabilities in patients are described throughout the application. In particular embodiments, the previously described technology discussed above can be applied to a broader patient population with implantable IVADs. Devices of the disclosure and those discussed above may include spectral imaging sensors that, when placed in contact with a patient's circulatory system or other bodily fluid, or skin, are capable of sensing one or more analytes to provide clinically actionable data related to the subject's heath status. In various embodiments, the data can relate to one or more of red blood cells, white blood cells, platelets, electrolytes, proteins, chemicals, drugs, circulating tumor cells, microbes, nucleic acids, and/or hemodynamics, and cardiac function, including blood flow rate and velocity and cardiac output of the subject, oxygen level, heart rate, body temperature, blood pressure, and ECG measurements of the patient.

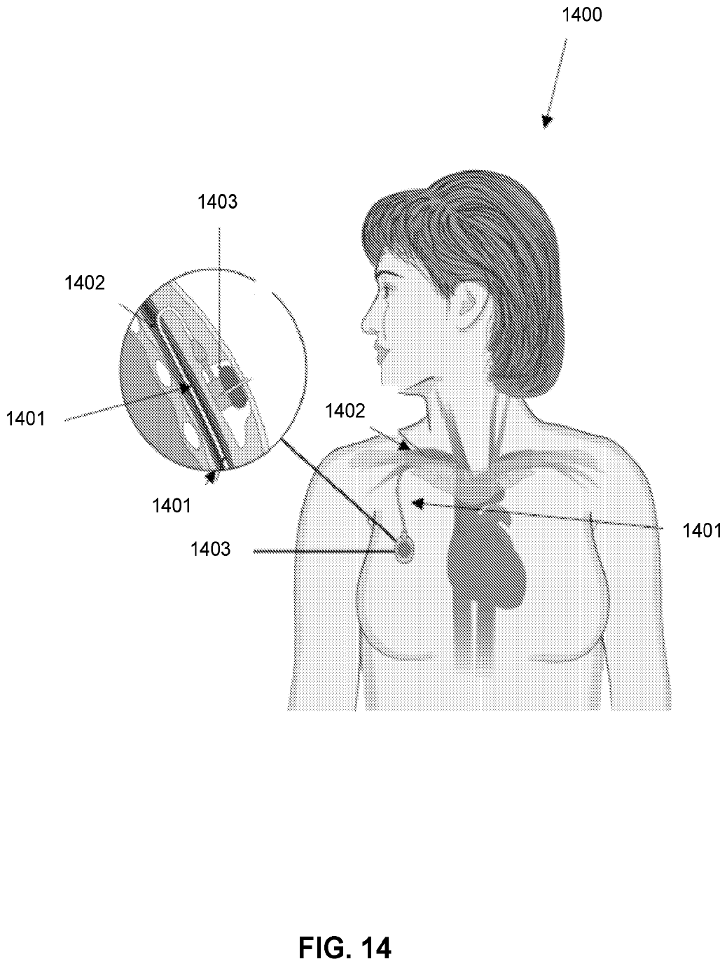

FIG. 14 shows an example a device 1400 implanted within a patient. The device 1400 includes a spectral imaging sensor system 1401 placed directly in a bloodstream via a catheter for measuring various analytes in the blood as it flows or circulates in the bloodstream, for example, through the superior vena cava 1402. Measurements are recorded from the blood and preferably transmitted, e.g., via a communication module, to one or more computing devices external to the subject. For example, the sensors may be configured to detect and measure various circulating elements in the bloodstream such as red blood cells, platelets, white blood cells, and/or other circulating components such as, for example, DNA, proteins, cancer cells. The port of the device 1403, which may be equipped with components described in U.S. patent application Ser. No. 16/932,503, is placed under the skin near a large vein in the upper chest.

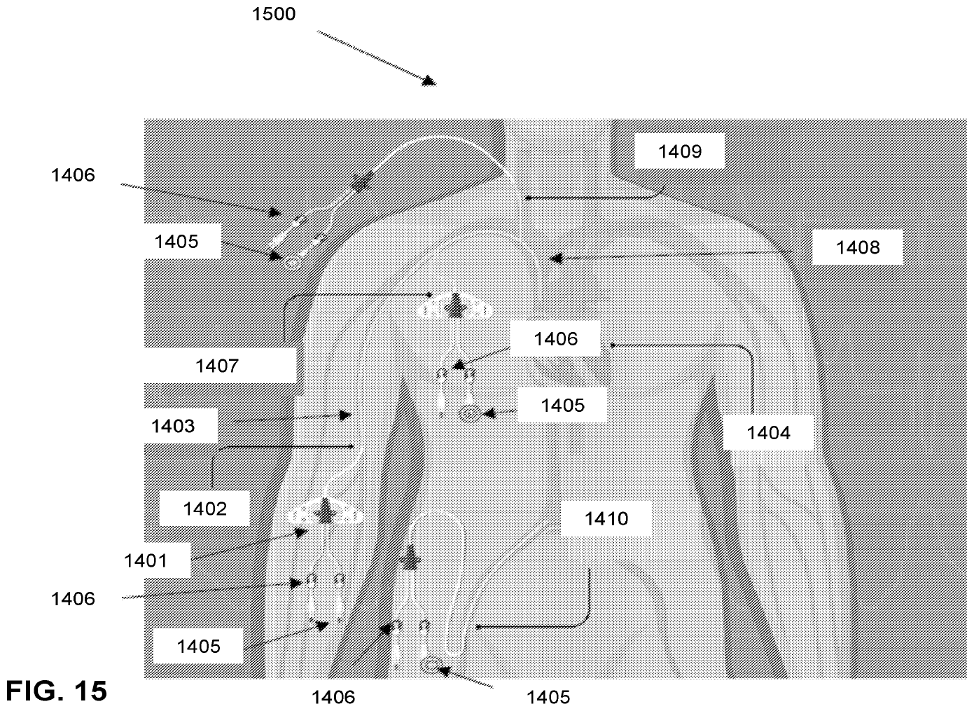

FIG. 15 depicts a number of different IVAD's that can be implanted in a subject 1500. A peripherally inserted central catheter (PICC) central venous catheter 1501 can also be used to administer therapy. A catheter 1502 is inserted into the vein of the arm 1503 until it reaches the heart 1504. PICC lines can be used for short-term (weeks) or longer term (months) use. The device 1501 has an external port 1505 with a single or double lumen, which may be equipped with the components described in U.S. patent application Ser. No. 16/932,503, and is attached to one end of the double-access catheter. In one embodiment, the loose device port is adhered to the skin of the clinical trial subject with surgical tape or similar. The catheter may also have clamps 1506 that provide access or close off access to the catheter. In some embodiments, a tunneled venous catheter 1507 is inserted into a central vein 1508, the jugular vein 1509 or the femoral vein 1510. In each type of central vein catheter an external device port 1505 with lumen, may be equipped with the components described in described in U.S. patent application Ser. No. 16/932,503, and attached to one end of the double-access catheter. In one embodiment, the loose device port is taped to the skin of the subject with surgical tape or similar. The catheters may also have clamps 1506 that provide access or close off access to the catheter.

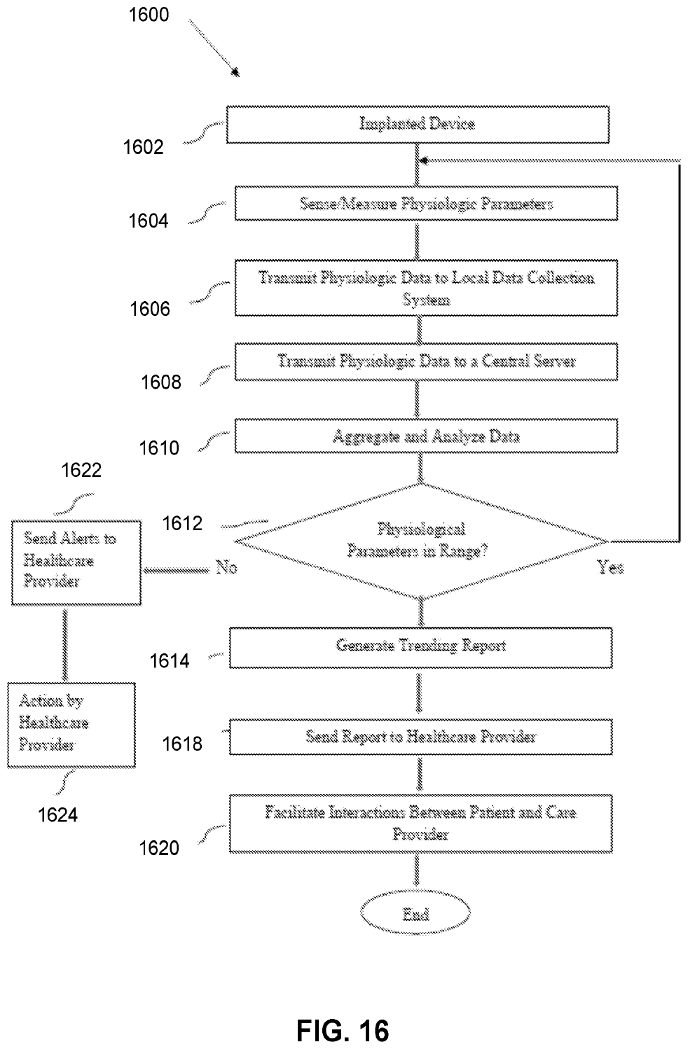

FIG. 16 shows a method 1600 for remote monitoring of a patient. The device, such as those described in U.S. Provisional Patent Application Ser. No. 63/174,319, can include a spectral imaging sensor system, configured to sense an analyte in the subject, which is implanted in the patient 1602. The method further includes measuring 1604 of an analyte by the sensor to generate data useful for assessing a health status of the subject. In further embodiments, the method 1600 transmits 1606 the physiological condition indicators/parameters to a local computing device which may also be referred to as a local data collection system. In some embodiments, the method 1600 can include transmitting 1608 the physiological condition indicators/parameters to a central server.

Assessing a health status may involve aggregating and analyzing the data 1610 generated by the sensing step. For example, analyzing 1610 may involve correlating signature profiles with signature profiles taken from a subject with a known health status. The methods of the invention can involve determining 1612 that the physiological condition indicators/parameters are within a predefined/expected range, then the method 1600 continues to measure 1604 one or more physiological condition indicators/parameters using one or more sensors integrated with the IVAD device.

If data is in range, the system may generate a trending report 1614 at intervals determined by the healthcare provider, and send the report 1618 to a treatment facility or the patient's health care provider. The report would facilitate interactions between the patient and his/her healthcare provider 1620. In certain embodiments, the remote monitoring system may be configured to send patient health alerts 1622 to one or more healthcare providers, such as, the subject's treating physician when physiologic parameters deviate from the set thresholds for a designated period of time. Alert messages are generated by the platform and sent to the healthcare providers via wireless communication links (e.g. email or text message) and facilitate action to be taken by the healthcare provider 1624, such as ordering of lab tests, blood transfusions or growth factors, and the method ends.

In some embodiments, data from the apparatus, system, and methods disclosed herein may provide data that upon proper authorization may be entered into a patient's electronic medical records (“EMR”). The apparatus, system, and methods described in the various embodiments above provide a number of important improvements over existing methods of monitoring physiological conditions of patients with IVAD's. For example, among the beneficial aspects of the apparatus, system, and methods described in the present disclosure and previous, are to monitor physiological functions in an integrated fashion and in real time and earlier evidence of physiological deviations to expected levels. This saves the time for the patient, physician, and laboratory time, as well as reducing overall costs to health care system.

Aspects of the invention may include a device with an implanted port-catheter or an implanted catheter with an external port, equipped with autofluorescence, multi-spectral (MSI) and hyperspectral imaging (HSI) sensor system to enable remote patient monitoring of health status. The device may be a port catheter or other IVAD, fitted for patients, implanted to enable infusion of a pharmaceutical, liquid or nutrient or withdraw blood or other body fluid. The device may measure an analyte in the patient by autofluorescence, MSI and HSI technologies. The analyte may be a blood cell count, a circulating tumor cells, a protein, a microbe, electrolyte, chemical, drug or a nucleic acid. The device may measure information related to one or more of red blood cells, white blood cells, platelets, circulating tumor cells, microbes, nucleic acids and/or hemodynamics, and cardiac function including, blood flow velocity and cardiac output, of the patient, oxygen level, heart rate, body temperature, blood pressure, and ECG measurements of the patient.

The device can measure chemicals and drugs in the blood or other body fluid. In certain embodiments, the device may measure blood pressure, body temperature, heart rate, heart function through an ECG, oxygen level, and electrolyte concentration from the skin. The sensor system (optical, thermal, or electronic, galvanic, impedance, amperometric) may collect data immediately upon implanting the device. The data collected by the device may be transmitted to a server or through an Industrial, Scientific and Medical (ISM) Band and analyzed covering 400 MHz to 2.4 GHZ radiofrequency spectrum. The data may be transmitted to a recipient such as a patient and/or Health Care Provider. The analyzed data may be shown to the recipients as a trending report. In some embodiments, if the analyzed data falls outside pre-specified ranges of normal, an alert can be sent to the health care team.

### Example 3—Use of Implantable Catheters for Assessing Health in Animals

The implantable medical devices described herein, having one or more spectral sensors for assessing a health status to enable remote health status monitoring, may be used animal subjects during preclinical and safety studies, and veterinary applications in an animal patient such as the treatment of cancer, pleurisy and/or other treatments or procedures requiring a port catheter or intravenous access device.

Animals are used, and often required by regulatory agencies, in medical device safety studies, pre-clinical trials and clinical trials. The animal model is based on the anatomy, physiology and application of the device. In addition, there are approved and available chemoport catheters and pleural ports for veterinary use in animal patients undergoing medical treatments. Animal health and stress monitoring is required to ensure the animal remains healthy and viable for treatment and studies. Remote patient monitoring provides the ability to monitor the animal patients and/or animal subjects, herein called animal(s), without frequent needle sticks, intervention and/or visits to or from the veterinarian to verify animal health status.

In certain embodiments, implantable devices with remote monitoring capabilities as described throughout the application may be used for assessing the health of an animal with an implanted chemo-port catheter, pleural port, or another implanted intravenous access device (IVAD). The implantable devices include one or more optical sensors that enable optical detection of one or more analytes inside the animal's body, using photonic means such as autofluorescence, multi-spectral (MSI) and hyperspectral imaging (HSI), covering the wavelengths from 400-2000 nm. For example, in preferred embodiments, the devices relate to catheters with one or more optical sensors capable of remotely measuring symptoms of physiological distress (e.g., body temperature, heart rate aberrations, blood cell count fluctuations) via the spectral sensors. In some embodiments, the sensor laden catheters are embodied in a port to deliver chemotherapy and/or other medical treatments and to monitor animal health over the course of treatment. Additional embodiments of the sensor laden catheter may be in the form of a peripherally inserted central catheter (also known as a PICC) or in the form of an implantable subcutaneous vascular access port (also known as a VAP) designed for repeated access into the animal's pleural cavity (also known as a pleural port) for treatments that require frequent blood monitoring and the delivery of fluids and medications. This disclosure incorporates an animal harness or collar uniquely designed to protect the device from environmental and animal damage, ensure the device and incision are maintained appropriately, and to allow ease of access when required while minimizing the discomfort to the animal.

As described in U.S. patent application Ser. No. 16/932,503 and throughout this application, spectral sensors can be used to sense physical light properties modulated (e.g., reflected, absorbed, dispersed, scattered, etc.) by one or more analytes present in the body to allow for earlier detection of signs indicative of a health status. This is useful for, among other things, early detection of complications associated with implantable devices, e.g., infections or thrombosis, to evaluate the animal's health prior to and/or as a result of treatment, and/or to provide recurrent health parameters during treatment regimens as prescribed by (pre)clinical trial protocols, study protocols and/or veterinarians.

Whereas implantable ports for animals exist, are considered effective long-term, and allow for the inclusion of sensors, they are not commonly used on animals. This is partly due to the fact that it is difficult to ensure the device is maintained in the manner required for safety, efficacy and effectiveness and partly due to the fact that treatments in animals prolong animal life but typically do not cure the disease state. Therefore, many pet owners opt to submit the animal to prescribed in-office tests and treatments and/or compassionately euthanize the animal. However, as treatment protocols become easier to deliver while maintaining the quality of life for the pet and/or as treatment regimens become more effective, it is likely pet owners will be more willing to use implantable ports, PICCs and similar. This device improves on the currently available veterinary products available for animals by enabling remote patient monitoring without human intervention, i.e., blood draws, veterinary visits, in addition to ensuring the device is safe and secure. This device also enables the pet and pet owner to maintain a close to normal life by ensuring the device is safe and secure in the home environment.

This device also improves and expands on the uses in preclinical/clinical trials and safety studies by incorporating sensors for remote patient monitoring into the product and protecting the product into a device specific harness or collar to prevent damage to the device. Animal studies and preclinical trials are required by regulatory agencies such as the U.S. Food and Drug Administration (FDA) and Health Canada before use in humans. It is essential to select an appropriate animal model that closely mimics the human disease conditions under study. Whereas many studies are initiated on rodents, studies often require expansion to or initiation on larger animals such as dogs, goats, porcine, that are physiologically closer to humans.

The data collected, and in the animal population described herein, may include, for example information related to one or more of red blood cells, white blood cells, platelets, proteins, electrolytes, circulating tumor cells, microbes, bacteria, nucleic acids, chemicals, drugs, oxygen and/or hemodynamics, and cardiac function including blood flow rate and velocity, and cardiac output, oxygen level, heart rate, body temperature, blood pressure, and ECG measurements of the animal In addition, the data collected may include: ECG, body temperature and heart rate.

Devices, as described in U.S. patent application Ser. No. 16/932,503 and throughout this disclosure, may include a spectral imaging sensor system configured to sense an analyte in the animal. The method further includes measuring of an analyte by the sensor to generate data useful for assessment of the health status of the animal. In further embodiments, the device can transmit the physiological condition indicators/parameters to a local computing device which may also be referred to as a local data collection system. In some embodiments, the device may transmit the physiological condition indicators/parameters to a central server. Assessing a health status may involve aggregating and analyzing the data generated by the sensing step. For example, analyzing the data may involve correlating signature profiles with signature profiles taken from an animal with a known health status. The methods of the invention can involve determining if the physiologic data from the animal are within expected and/or predetermined ranges or outside of expected physiological condition indicators/parameters. The method may allow alert limits to be identified by the (pre)clinical protocol/investigator, healthcare provider and/or veterinarian in order to facilitate prompt and appropriate treatment/animal care, herein called appropriate care giver(s). The generated alert message will be sent to the appropriate care giver(s) which may include the animal owner as owners often participate in the monitoring and treatment of the animal. In addition, the system may generate a report at defined intervals or upon an alert and send the report to the appropriate care giver(s) including but not exclusive to the veterinarian, the lead investigator, the company sponsor and the animal owner, herein referred to as the care team or care team member(s). The report can facilitate interactions with all appropriate personnel involved. In some embodiments, data from the apparatus, system, and methods disclosed herein may provide data that upon proper authorization may be entered into the animal's electronic medical records (“EMR”). As required, the data may be de-identified prior to submission in order to support blinded clinical trial requirements which is not typical in animals but is occasionally required.

Port and catheter sizes depend on the animal size and type of animal. For example, animal devices may be available in the following configurations:

Small: ferrets, small cats, or similar—3.5 French catheter, small access port Medium: cats, small dogs, or similar—5.0-7.0 French catheter, medium access port Large: large dogs, or similar—7.0-9.0 French catheter, large access port Devices may be configured in smaller and/or larger configurations as determined appropriate for the type of animal and as available. These typically range in size from 1 French for a small rodent such as a mouse up to and beyond 16 French for very large animals. As the length of the catheter is typically sized to the specific animal, this product may be embodied in one of the following manners: a) the sensors may be molded into the catheter such that the catheter may be cut to size without damage to the sensors where the sensors are located in a clinically relevant location but distal to the catheter tip, b) the device will have various pre-sized catheter lengths, or c) the device will have a dual lumen catheter where the sensor bundle will be threaded through the secondary lumen to desired length allowing for shortening and attachment at the port. The implant time varies depending on the treatment, which can range from days (short-term), up to 6 months (intermediate-term) or 6 months or longer (long-term).

As described in U.S. patent application Ser. No. 16/932,503 and throughout the present disclosure, the devices of the invention may be equipped with wireless communication components that provide for remote transmission of clinically actionable data to one or more locations, such as, study location, veterinarians and/or care provider. The catheter device may further include a port having a self-sealing septum for receiving fluid (e.g., a chemotherapy agent) via a needle. In the embodiment of a pleural port, it will also contain a non-coring needle known as a Huber point needle, to support the drainage of fluid.

Implantable intravascular devices having remote physiological monitoring capabilities in patients are discussed throughout the disclosure. In certain embodiments, these devices and methods may be applied in animals with implantable IVADs. Devices of the disclosure may include spectral imaging sensors that, when placed in contact with an animal's circulatory system or other bodily fluid, or skin, are capable of sensing one or more analytes to provide clinically actionable data related to the animal's heath status. In particular, devices of the invention interrogate tissue (e.g., blood) with short pulses of blue, green, red, or infrared light, or a broad spectral white light. The light is preferably delivered by one or more thin optical fibers of the implantable device. The light is manipulated (e.g., dispersed, scattered, absorbed, etc.) by one or more analytes present in tissue. The light manipulations may be sensed by a second fiber or bundle of fibers adjacent to the first fiber, to thereby collect data related to the one or more analytes and provide clinically useful information. The information may relate to one or more of red blood cells, white blood cells, platelets, electrolytes, proteins, chemicals, drugs, circulating tumor cells, microbes, nucleic acids, and/or hemodynamics, and cardiac function including, blood flow rate and velocity and cardiac output, oxygen level, heart rate, body temperature, blood pressure, and electrocardiogram ECG measurements of the animal.

This information is useful to identify, among other things, early signs that an animal may be suffering from an infection and/or that a treatment is producing dangerous side effects (e.g., organ failure). In other instances, this information may be useful for showing signs of disease, for example, a genetic disease, an autoimmune disease, a neurologic disease, a metabolic disease, or a chemotherapy related condition.

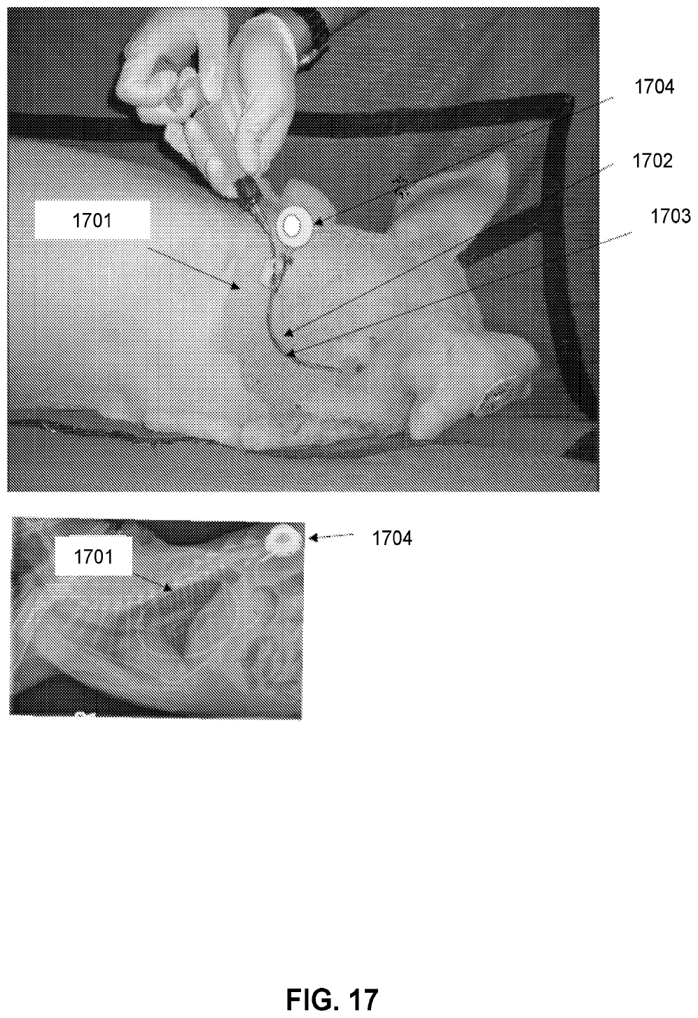

FIG. 17 shows a device 1701, from an external and internal view, which includes a spectral imaging sensor system 1702 placed directly in a bloodstream via a catheter for measuring various analytes in the blood as it flows or circulates in the bloodstream, for example, through the central vein 1703. Measurements are recorded from the blood and preferably transmitted, e.g., via a communication module, to one or more computing devices external to the subject. For example, the sensors may be configured to detect and measure various circulating elements in the bloodstream such as red blood cells, platelets, white blood cells, and/or other circulating components such as, for example, DNA, proteins, cancer cells. The port of the device 1704, which may be equipped with components described in U.S. patent application Ser. No. 16/932,503, is placed under the skin near a large vein in the upper chest. In one embodiment, the port is external and taped to the skin of the animal with surgical tape, and/or protected and enclosed in a harness or collar designed for the device.

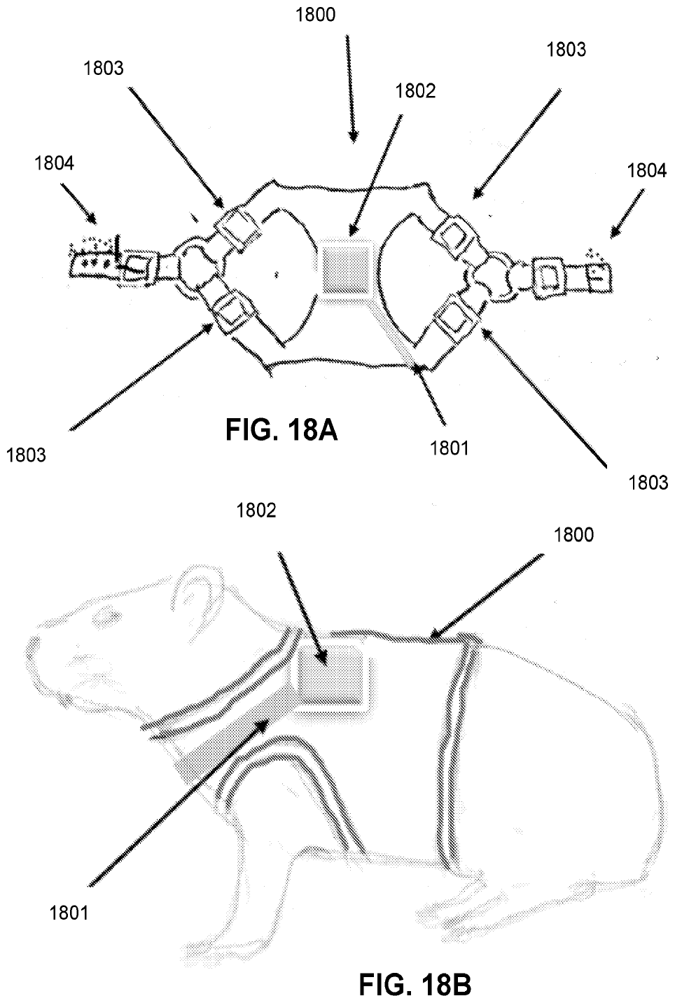

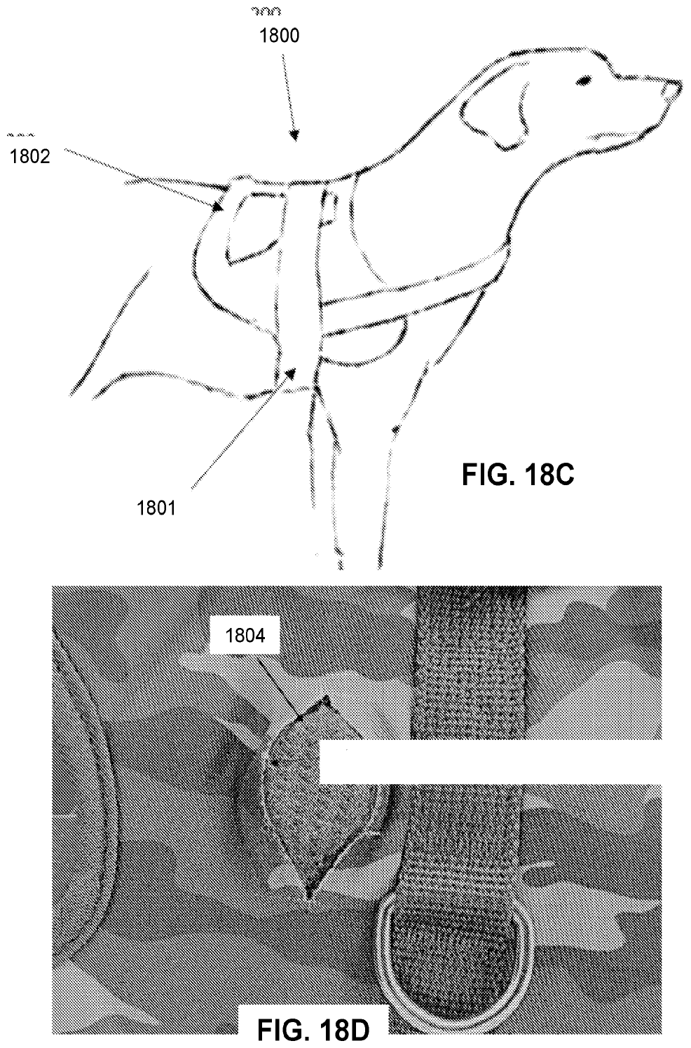

FIGS. 18A through 18D depicts examples of device harnesses for animals 1800 that come in various animal sizes. The harness has extra protection to protect the ports and catheters 1801 as well as the incision on the animal. The upper box 1802 depicts the protective pocket inside the harness that allows any portion of the device that extends outside the body to be safely tucked in when not in use and allows easy access to the implanted septum for delivery and/or removal of fluids. The harness may have adjustable leg straps or pre-sized openings 1803 or pre-sized openings for common sized animals. The harness may have a Velcro, or similar, closure at the back of the harness 1804. FIG. 18D depicts a closeup of the opening of the protective pocket for easy access to the port and/or catheter openings.

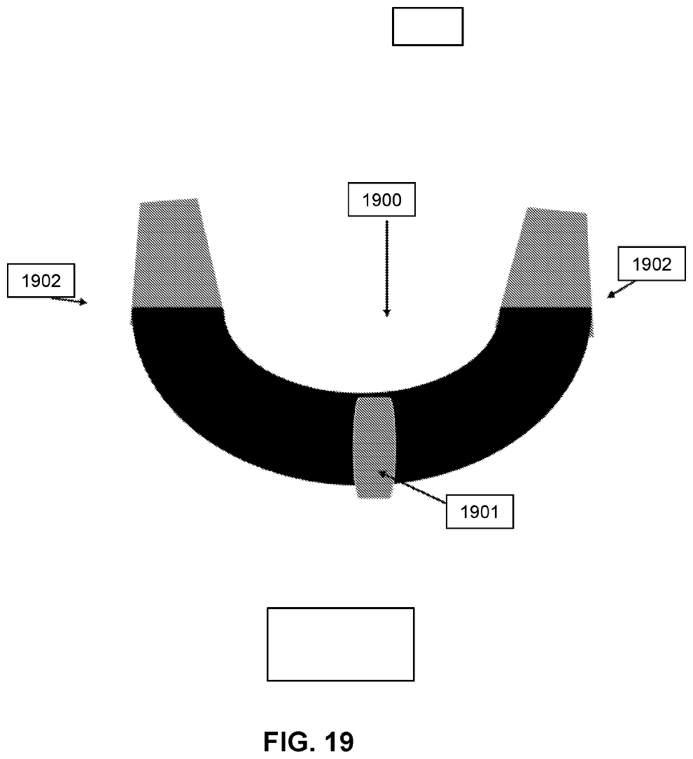

FIG. 19 depicts a device collar 1900 for larger animals such as cows, goats and other animals that can wear a wide collar without being able to remove it. The collar, similar to the harness, is designed to protect the ports and the catheters as well as the incision on the animal. A Velcro closured protective pocket 1901 inside the collar can allow any portion of the device that extends outside the body to be safely tucked in when not in use and/or easy access to the port septum without removal of the collar. The collar has adjustable straps or Velcro 1902 to ensure the collar fits the animal appropriately.

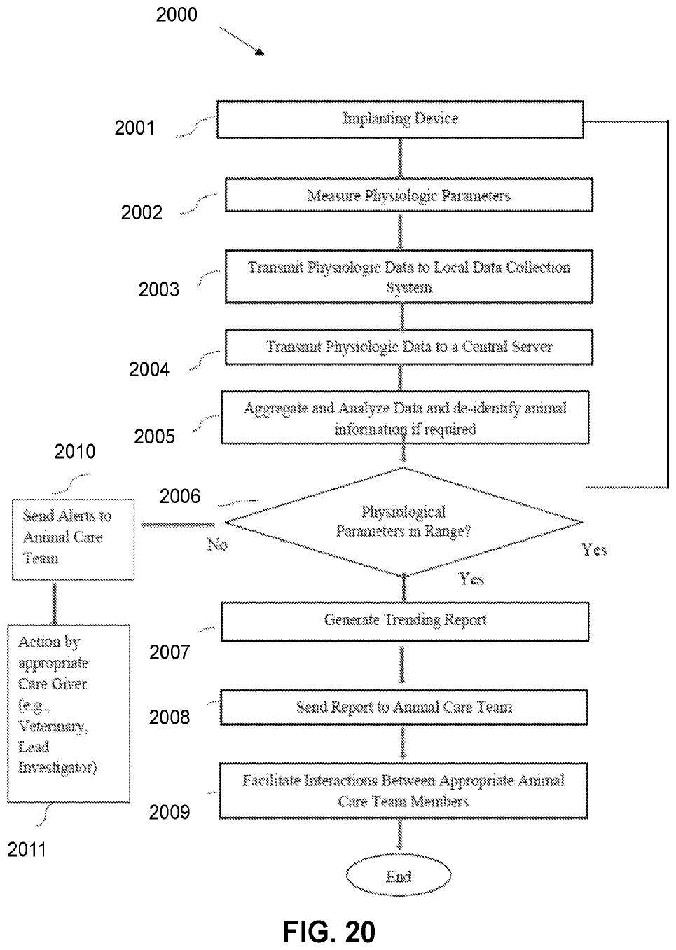

FIG. 20 shows a method 2000 for remote monitoring of animal health. The device, such as those described throughout the present disclosure, can include a spectral imaging sensor system configured to sense an analyte which is implanted in the animal 2001. The method further includes measuring 2002 of an analyte by the sensor to generate data useful for assessing a health status of the animal. In further embodiments, the method 2000 may include transmitting 2003 the physiological condition indicators/parameters to a local computing device which may also be referred to as a local data collection system. In some embodiments, the method 2200 may include transmitting 2204 the physiological condition indicators/parameters to a central server. Assessing a health status may involve aggregating and analyzing the data 2205 generated by the sensing step. For example, analyzing 2205 may involve correlating signature profiles with signature profiles taken from a subject with a known health status. In some cases, unique animal identification information may be de-identified, if animal is subject to a clinical trial protocol. The methods of the invention involve determining if the physiologic data is in 2200 determines 2206 that the physiological condition indicators/parameters are within a predefined/expected range, then the method 2200 continues to measure 2202 one or more physiological condition indicators/parameters using one or more sensors integrated with the implantable VAP device.

If data is in range, the system may generate a trending report 2207 at intervals determined by the care giver(s) and send the report 2208 to the animal care team. The report would facilitate interactions between the appropriate animal care team members 2209. In certain embodiments, the remote monitoring system may be configured to send health alerts 2210 to one or more of the care team, such as, the animal's veterinarian when physiologic parameters deviate from the set thresholds for a designated period of time. Alert messages are generated by the platform and sent to the care giver(s) via wireless communication links (e.g., email or text message) and facilitate action to be taken by the appropriate care giver 2211, such as ordering of lab tests, administering fluids or medications, and the method 2200 ends. In some embodiments, data from the apparatus, system, and methods disclosed herein may provide data that upon proper authorization may be entered into an animal's electronic medical records (“EMR”).

Aspects of the invention may include a device with an implanted port-catheter or an implanted catheter with or without an external port equipped with the autofluorescence, multi-spectral (MSI) and hyperspectral imaging (HSI) sensor system to enable remote patient monitoring of animal health status. The device may be an implanted catheter fitted for animals to enable infusion of a pharmaceutical and/or removal of fluid. The device may be capable of measuring an analyte in the animal by autofluorescence, MSI and HSI technologies. The analyte may be a blood cell, a circulating tumor cell, a protein, a microbe, chemical, drug or a nucleic acid.

In certain embodiments, the device may measure blood cell count, blood flow rate and blood flow velocity. The device can measure blood pressure, body temperature, heart rate, oxygen level, heart function through an ECG and electrolyte concentration from the skin. The sensor system (optical, thermal, or electronic, galvanic, impedance, amperometric) may collect this data immediately upon implanting the device. The data collected by the device may be transmitted to a server or through an Industrial, Scientific and Medical (ISM) Band and analyzed covering 400 MHz to 2.4 GHz radiofrequency spectrum.

The analyzed data may be transmitted to a care team or care team member such as a veterinarian, preclinical trial manager, personnel responsible for the animal study and/or animal owner to track. In certain embodiments, the analyzed data may be shown in the form of trending report. In some embodiments, if the analyzed and/or trended data falls outside of pre-specified ranges of normal, an alert may be sent to the appropriate care team member(s) as indicated in

The software that transmits the data may enable the animal identification information to be de-identified or blinded from recipients to support clinical trial protocols requiring blinded data and supports the ability to unblind the data and ensure all data can be identified to correct animal when required, through access control. The software that transmits the data may allow for auditing and traceability capabilities and control. The device may be protected by a specialized harness or collar to prevent the animal from damaging the device and/or injuring their incision created during the device placement and to minimize the potential for infection at the incision site.

### Example 4—Use of Implantable Spectral Imaging System for Assessing Health Status Remotely of Subjects Enrolled in Clinical Trials

The implantable medical devices described herein, having one or more spectral sensors for assessing a health status to enable remote health status monitoring, may be used to monitor the health status of subjects enrolled in clinical trials in which an implanted intravascular access device may be indicated.

Subjects recruited and enrolled in clinical trials, especially in the field of oncology, may be indicated to receive an implantable intravenous access device (IVAD) either centrally or peripherally implanted. In addition, there are multiple clinical trials in which various IVAD's are under study. IVAD's are used in a variety of investigative settings to assist with drug delivery and sampling of blood over the course of a clinical trial. Clinical trials span a variety of diseases, including studies to investigate oncology drugs, metabolic diseases, cardiovascular diseases and hemodialysis, and others that have implanted catheters and require health monitoring. In the example of oncology drug clinical trials, subjects may have a chemo-port catheter implanted in their chest, with the catheter inserted into the subject's superior vena cava, enabling access to the heart and blood. Many chemotherapeutic agents to treat breast cancer and other solid tumors are given intravenously, such as carboplatin, cyclophosphamide (oral or intravenously), doxorubicin, epirubicin, fluorouracil, gemcitabine, paclitaxel, vincristine, ixabepilone, cisplatin and docetaxel. Lymphomas and leukemias are also often treated with infused cancer chemotherapeutic agents and antibody therapies as well, and include carboplatin, cladribine, daunorubicin, ibritumomab and rituxan. In addition, immunotherapeutic agents and antibody-related therapies such as nivolamab, Rituxan, daratumamab, darzlex elotuzumab, remicade, to name a few, are also infused. Although the drugs mentioned are currently approved, they are still used in clinical trials to investigate combination therapies or new formulations, indications and dosing regimens. Approved drugs in clinical trials or drugs under investigation have associated toxicities, including neutropenia, thrombocytopenia, leukopenia, fever, cardiotoxicity, and bone marrow suppression.

During the course of clinical trials, subjects may experience a variety of complications and adverse events, along with their clinical response (positive or negative) to the investigative drug. Subjects are monitored periodically throughout the clinical trial. Clinical trial sponsors and principal investigators capture a host of information from clinical trial subjects during their drug and after their studies, which may include in person physical assessments, laboratory tests, body scans and imaging technologies as outpatients, as well as remote patient monitoring technologies. In some cases, subjects are also asked to report symptoms to their clinician, nurse or the principal investigator of the study. Patient Reported Outcomes (PRO's), such as disease symptoms, symptomatic adverse events and physical function, can be subjective and qualitative, and oftentimes may not reflect physiologic changes occurring in the body. In addition, de-centralized clinical trials are increasingly used as an option to improve subject recruitment and diversity across wider geographic areas. Decentralized clinical trials have many benefits, including reduced burden to subjects and study sponsors. Whether centralized or decentralized, modifications of trial designs such as inclusion of remote assessments, are more frequent to reduce subject's exposure COVID-19 or other infectious agents posing risks to immunocompromised populations. Studies have shown subjects who are of low-income, rely on public transportation, disabled, or have to drive more than 20 miles for treatments while enrolled in clinical trials, are especially challenged and are at higher risk for serious adverse events. This invention describes the ability to monitor clinical trial subjects, remotely and passively, for complications and outcome analyses, by measuring physiologic functions through an implantable port-catheter or other implanted intravenous access device equipped with optical sensor technology. The ability to monitor subjects passively during the course of the clinical trial, will enable principal investigators and pharmaceutical companies to obtain assess the health status of clinical trial subjects more frequently and without their reducing the need to travel. This data may be useful post clinical trial outcome analysis, for example in sensitivity analysis, drug monitoring, predicting drug response and predicting adverse reactions.

Systems and methods of the invention can address these issues by providing implantable devices (e.g, ports) with remote monitoring capabilities for assessing health status of a subject enrolled in a clinical trial. This disclosure provides, for example, the use of implantable devices with remote monitoring capabilities for assessing health of a patient with an implanted chemo-port catheter or another implanted intravenous access device (IVAD), such as a peripherally inserted central catheter (PICC) line.

Devices used for remote monitoring of subjects in a clinical trial may include any of the features described throughout the application. Catheter devices may further include a port having a self-sealing septum for receiving fluid (e.g., a chemotherapy agent) via a needle and monitor subject health over the course of the clinical trial. In some embodiments, IVAD's utilized in non-cancer disease states, such as endocrine, cardiovascular and autoimmune diseases, can also be equipped with optical sensors to monitor health status of subjects enrolled in clinical trials in which IVAD's are used.

As described in U.S. patent application Ser. No. 16/932,503 and throughout this disclosure, spectral sensors can sense physical light properties modulated (e.g., reflected, absorbed, dispersed, scattered, etc.) by one or more analytes present in the body to allow for earlier detection of signs indicative of a health status. Identification and count of prokaryotic cells, including blood cells, tumor cells, fibroblasts, etc. and molecular analytes can be further characterized by use of spatial profiling via HSI. Use of photonic sensing technology can also be applied to detection of eukaryotic cells such as fungi and bacteria. This is useful for, among other things, early detection of complications associated with implantable ports, e.g., infections or thrombosis, and to evaluate subjects during a clinical trial in which an IVAD or investigational drug is under study.

Advantages of using devices and methods of the invention during clinical trials may include:
1) Reducing the number of needles sticks for medication, fluids, nutrition, blood products and blood samples during clinical trials.
2) Reducing the irritation of veins exposed to drugs.
3) Allowing for the ability to infuse multiple types of medication at the same time in combination clinical trials.
4) Enabling more frequent monitoring of health status for subjects enrolled in clinical trials, reducing attrition rate or rate of acute complications.
5) Enabling more convenient health assessment of subjects, especially those enrolled in de-centralized clinical trials, and reducing the need to travel to assessment sites or test labs.
6) Allowing for the ability to enroll a broader spectrum of subjects due to the ability to monitor the subjects remotely.

In some embodiments, devices of the invention can include an assembly of components for detection, analysis, and transmission of clinically relevant data.

As described in U.S. patent application Ser. No. 16/932,503 and throughout the present disclosure, the devices of the invention may be equipped with wireless communication components that provide for remote transmission of clinically relevant data to one or more locations, such as, one or more treatment facilities, the principal investigator and/or clinician. For example, devices of the invention may include a two-dimensional photosensor array and a fiber optic image conduit. The fiber optic image conduit may be operable to relay data from the spectral imaging sensor system to the two-dimensional photosensor array. Preferably, the two-dimensional photometric sensor array is operable to acquire data at a high sampling rate of more than 10 frames per second. Devices may further include a broad-spectral light source and a communication module operable to provide data to a computing device that is external to the subject. In some embodiments, the assembly of these components will be designed into the port of a chemo-port which is implanted subcutaneously in the subject. In some embodiments, the port may not be implanted subcutaneously, but rather attached to the end of one of the catheter lumens accessible outside the body, and held into place with surgical tape, arm band, chest band or similar.

The data collected in the clinical trial subjects may include, for example, information related to one or more of red blood cells, white blood cells, platelets, proteins, electrolytes, circulating tumor cells, microbes, nucleic acids and/or hemodynamics, and cardiac function including, blood flow rate and velocity and cardiac output, oxygen level, heart rate, blood pressure, body temperature and ECG measurements of the clinical trial subject. In some embodiments, sensor data may include identifiable chemicals or drugs for use in clinical pain management applications.

As previously described in U.S. patent application Ser. No. 16/932,503 and throughout the present disclosure, certain aspects of the invention may be useful to address complications that arise in subjects during drug and device clinical trials. Adverse events can occur during clinical trials, especially in oncology. Subjects are often ill already, and further can be immunosuppressed and traveling to and from doctor's offices. These circumstances can increase the likelihood of a serious adverse event (SAE). Subject evaluations are often performed at clinical facilities prior to certain treatments, such as, chemotherapy treatments. These evaluations may be the first evaluation that the subject has received in an extended period of time (e.g., since the subject's last visit to the infusion center). Often, the evaluations reveal that it is unsafe to proceed with therapy due to uncontrolled symptoms, such as, low blood counts, or fever. This results in delays in the experimental treatment, and attrition of subjects enrolled in clinical trials. Such delays and attrition, however, may be avoidable by implementing in vivo remote assessment devices provided by this disclosure.

Implantable intravascular devices having remote physiological monitoring capabilities in adult patients undergoing treatment for cancer or other conditions are discussed throughout the present disclosure. In certain embodiments, any of the aforementioned technology may be applied to human subjects enrolled in clinical trials to investigate infused therapies or combinations wherein in which a chemo-port or other IVAD is indicated, or in clinical trials investigating new or improved IVAD's. Devices of the disclosure and those cross-referenced include spectral imaging sensors that, when placed in contact with a patient's circulatory system or other bodily fluid, or skin, are capable of sensing one or more analytes to provide clinically actionable data related to the subject's heath status. In particular, devices of the invention may interrogate tissue (e.g., blood) with short pulses of blue, green, red, or infrared light, or a broad spectral white light. The light is preferably delivered by one or more thin optical fibers of the implantable device. The light is manipulated (e.g., dispersed, scattered, absorbed, etc.) by one or more analytes present in tissue. The light manipulations may be sensed by a second fiber or bundle of fibers adjacent to the first fiber, to thereby collect data related to the one or more analytes and provide clinically useful information. The information may relate to one or more of red blood cells, white blood cells, platelets, electrolytes, proteins, chemicals, drugs, circulating tumor cells, microbes, nucleic acids, and/or hemodynamics, and cardiac function including, blood flow rate and velocity and cardiac output, oxygen level, heart rate, body temperature, blood pressure, and ECG measurements of the subject. This information is useful to identify, among other things, early signs that a subject may be suffering from an infection and/or that a treatment is producing dangerous side effects from investigational drug treatment or medical devices.

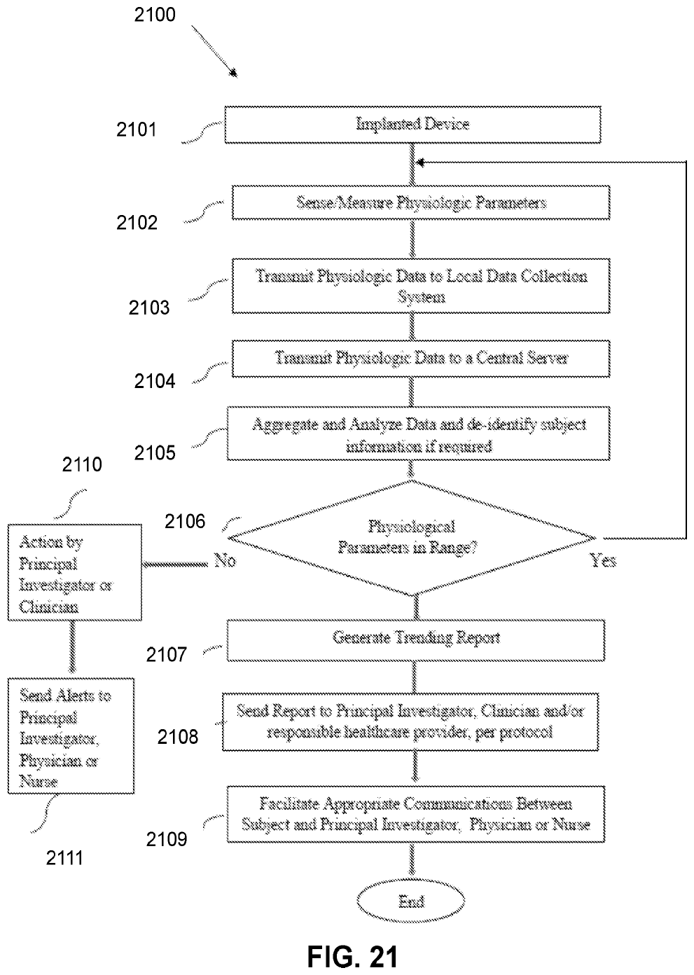

FIG. 21 shows a method 2100 for remote monitoring of subject's health. The device, as described above, may include a spectral imaging sensor system configured to sense an analyte in the subject, which is implanted in the pediatric patient 2101. The method further includes measuring 2102 of an analyte by the sensor to generate data useful for assessing a health status of the subject. In further embodiments, the method 2100 can include transmitting 2103 the physiological condition indicators/parameters to a local computing device which may also be referred to as a local data collection system. In some embodiments, the method 2100 may include transmitting 2104 the physiological condition indicators/parameters to a central server. Assessing a health status may involve aggregating and analyzing the data 2105 generated by the sensing step.

For example, analyzing 2105 may involve correlating signature profiles with signature profiles taken from a subject with a known health status. In some cases, patient information would be de-identified, if required by the clinical trial protocol.

The methods of the invention involve determining if the physiological condition indicators/parameters are within a predefined/expected range, then the method 2100 continues to measure 2102 one or more physiological condition indicators/parameters using one or more sensors integrated with the IVAP device.

If data is in range, the system may generate a trending report 2107 at intervals determined by the healthcare provider, and send the report 2108 to a treatment facility or the patient's guardian. The report would facilitate interactions between the subject and healthcare clinician, Principal Investigator (PI) or nurse 2109.

In certain embodiments, the remote monitoring system may be configured to send patient health alerts 2110 to one or more healthcare providers, such as, the subject's treating physician when physiologic parameters deviate from the set thresholds for a designated period of time. Alert messages are generated by the platform and sent to the healthcare providers via wireless communication links (e.g. email or text message) and facilitate action to be taken by the clinician or study nurse 2111, such as ordering of lab tests, blood transfusions or growth factors, and the method ends.

In some embodiments, data from the apparatus, system, and methods disclosed herein may provide data that upon proper authorization may be entered into the clinical trial database. As required, the data may be de-identified prior to submission in order to support blinded clinical trial requirements. The apparatus, system, and methods described in the various embodiments above provide a number of important improvements over existing methods of monitoring physiological conditions of pediatric patients with IVAD's. For example, among the beneficial aspects of the apparatus, system, and methods described in the present disclosure and previous, are to monitor physiological functions in an integrated fashion and in real time and provide early evidence of adverse events. This saves the time for the subject, clinician, nurse, sponsor, as well as reducing overall costs to clinical trial by the study sponsor and health care system.

Aspects of the invention may include a device with an implanted port-catheter or an implanted catheter with or without an external port, equipped a with an autofluorescence, multi-spectral (MSI) and hyperspectral imaging (HSI) sensor system to enable remote patient monitor of health status of subjects enrolled in centralized or decentralized clinical trials. The device may be a port catheter or other IVAD, implanted to enable infusion of an investigational pharmaceutical, administered alone or in combination with another therapy, or a pharmaceutical that is standard of care. The device can be a port catheter or other implantable catheter under investigation in the clinical trial. The device can measure an analyte in the clinical trial subject by autofluorescence, MSI and HSI technologies. The analyte may be a blood cell, a circulating tumor cell, a protein, a microbe, electrolyte, chemical, drug or a nucleic acid.

In certain embodiments, the device may measure information related to one or more of red blood cells, white blood cells, platelets, circulating tumor cells, microbes, nucleic acids and/or hemodynamics, and cardiac function including, blood flow velocity and cardiac output, oxygen level, heart rate, body temperature, blood pressure, and ECG measurements of the patient. The device can measure chemicals and drugs in the blood or other body fluid. The device may measure blood pressure, body temperature, heart rate, heart function through an ECG, oxygen level, and electrolyte concentration from the skin. The sensor system (optical, thermal, or electronic, galvanic, impedance, amperometric) may collect this data immediately upon implanting the device. The data collected by the device can be transmitted to a server the data collected by the device is transmitted to a server or through an Industrial, Scientific and Medical (ISM) Band and analyzed covering 400 MHz to 2.4 GHz radiofrequency spectrum. The analyzed data may be transmitted to a recipient such as a Principal Investigator, clinician, or personnel as designated by the clinical trial protocol. The analyzed data can be presented to the recipient as a trending report.

If the analyzed data falls outside pre-specified ranges of normal, an alert can be sent to a designated recipient, such as the principal investigator or subject's primary care physician. The software associated with transmitting the data may de-identify the data or otherwise cause the data to be blinded from recipients to support clinical trial protocols requiring blinded data and support the ability to unblind the data and ensure all data can be identified to correct subjects when required, through access control. Software associated with transmitting the data may allow for auditing and traceability capabilities and control.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. An implantable device comprising a multi-spectral imaging sensor system.

2. The implantable device of claim 1 wherein the multi-spectral imaging sensor system comprises one or more optical thermal, electronic, galvanic, impedance, or amperometric sensors.

3. The implantable device of claim 1, wherein the multi-spectral imaging sensor system comprises a spectrum-resolving component comprising at least one of a filter, a grating, or a prism, wherein the spectrum-resolving component is operable to distinguish a plurality of distinct wavelengths of light between 400 and 2000 nanometers.

4. The implantable device of claim 1, further comprising at least one additional component operable to sense autofluorescence.

5. The implantable device of claim 1, further comprising multi-spectral imaging and hyperspectral imaging technologies.

6. The implantable device of claim 1, wherein the device is a port catheter comprising a cannula, and wherein the multi-spectral imaging sensor system is attached to a distal portion of said cannula.

7. The implantable device of claim 1, wherein the device is a PICC line fitted for pediatric patients, implanted to enable infusion of a pharmaceutical or withdraw blood.

8. The implantable device of claim 1, wherein the device is a tunneled catheter line fitted for pediatric patients, implanted to enable infusion of a pharmaceutical or withdraw blood, or to administer liquid nutrition.

9. The implantable device of claim 1, wherein the device is a catheter fitted for animals to enable infusion of a pharmaceutical and/or removal of fluid.

10. The implantable device of claim 1, wherein the device is operably associated with a machine learning system.

11. The implantable device of claim 1, further comprising a two-dimensional photosensor array and a fiber optic image conduit, wherein the fiber optic image conduit is operable to relay data from the multi-spectral imaging sensor to the two-dimensional photosensor array.

12. The implantable device of claim 11, wherein the two-dimensional photometric sensor array is operable to acquire data at a high sampling rate of more than 10 frames per second.

13. The implantable device of claim 1, further comprising a broad-spectral light source.

14. The implantable device of claim 1, further comprising a communication module operable to provide data to a computing device that is external to the subject.

15. The implantable device of claim 14, wherein the communication module is operable to provide data to a health care provider or animal care provider.

16. The implantable device of claim 1, configured to remain in place for at least one week.

17. The implantable device of claim 1, wherein the device is protected by a specialized harness or collar, wristband, or chest band to protect the a multi-spectral imaging sensor system when implanted in a subject or animal.

18. A method to monitor patient health, the method comprising: implanting a device into a subject, the device comprising a multi-spectral imaging sensor system configured to sense an analyte in the subject, wherein sensing of the analyte by the sensor is used to assess a health status of the subject.

19. The method of claim 18, wherein the analyte comprises a blood cell, a circulating tumor cell, a protein, a microbe, an organic compound, a chemical, a chemical composition, a drug, or a nucleic acid.

20. The method of claim 18, wherein the device is further configured to measure one or more of blood pressure, body temperature, heart rate, heart function through an ECG, oxygen level, and electrolyte concentration from the skin.

21. The method of claim 18, further comprising sensing, with the multi-spectral imaging sensor, one or more of light dispersion, light scattering, light diffraction, or light interference.

22. The method of claim 21, wherein light sensed by the multi-spectral imaging sensor is useful to assess one or more of size, granularity, nuclear size, shape, organic chemical composition, or cytoplasmic density, of the analyte.

23. The method of claim 22, wherein the multi-spectral imaging sensor system comprises spectrum-resolving components, wherein the spectrum resolving components comprises one or more of, a filter, a grating, or a prism for sensing a plurality of distinct wavelengths of light between 400 and 2000 nanometers, and wherein the plurality of distinct wavelengths of light are indicative of one or more properties of the analyte.

24. The method of claim 18, wherein the sensor collects data immediately upon implanting the device.

25. The method of claim 18, wherein the device further comprises at least one autofluorescence, multi-spectral, or hyperspectral imaging sensor.

26. The method of claim 18, wherein the device further comprises a catheter comprising a port subcutaneously implanted or held in place external to the body and connected to a reservoir for receiving fluid by a needle.

27. The method of claim 18, wherein the device, when implanted, extends into at least one of a superior vena cava, a right atrium, a peripheral vein or artery, or a central vein or artery.

28. The method of claim 18, wherein the device is configured to assess the health status of the subject remotely.

29. A method for collecting research or clinical data, the method comprising: receiving, at a device, data based on light sensed by a multi-spectral imaging sensor system implanted in a subject.

30. The method of claim 29, wherein the data is provided from a remote location by a wireless data network.

31. The method of claim 29, wherein the data comprises information related to one or more of red blood cells, white blood cells, platelets, circulating tumor cells, microbes, organic compounds, chemicals, chemical composition, drugs, nucleic acids and/or hemodynamics, and cardiac function including blood flow rate, velocity and cardiac output, including blood flow rate, velocity, oxygen level, heart rate, body temperature, blood pressure, and electrocardiogram (ECG) measurements of the subject.

32. The method of claim 29, wherein the data comprises multi-spectral light dispersion, scattering, absorption data of cells circulating in a blood stream of the subject.

33. The method of claim 29 wherein the data collected by the device is transmitted to a server or through an Industrial, Scientific and Medical (ISM) Band and analyzed covering 400 MHz to 2.4 GHz radiofrequency spectrum.

34. The method of claim 29, further comprising analyzing the data to generate an assessment of the subject's health.

35. The method of claim 34 in which the analyzed data is shown to the recipients as a trending report.

36. The method of claim 34, further comprising providing an alert to a physician based on the assessment of the subject's health.

37. The method of claim 34, wherein analyzing involves correlating the data from the subject with pre-determined parameters associated with a physiological condition.

38. The method of claim 37, wherein the physiological condition comprises one of a genetic disease, an autoimmune disease, a neurologic disease, a metabolic disease, or a chemotherapy related condition.

39. The method of claim 37, wherein the parameters are established based on input of a health care professional.

40. The method of claim 34, wherein the assessment is used to identify the subject as needing a treatment.

41. The method of claim 34, further comprising providing said assessment to a health care professional and/or the subject.

42. The method of claim 34, wherein the assessment is indicative of a change in the subject's health.

43. The method of claim 34, further comprising de-identifying or blinding the subject from recipients.

44. The method of claim 43, wherein the subject is a clinical trial participant.

45. The method of claim 43, further comprising providing access control to unblind the data.

46. The method of claim 29, further comprising providing data auditing and/or tracing capabilities for received data.

47. The method of claim 29, wherein the subject is a patient undergoing treatment, an animal, a pediatric patient, or a clinical trial subject.

48. The method of claim 47, wherein the animal comprises one of a pet, a non-human primate, a research animal, a horse, or a cow.
